SEQUENCE LISTING (254 sequences)

SEQ ID NO: 1 - RNA, 19 nt, artificial sequence
  inhibitory RNA
auaacucuguccauuaccg

SEQ ID NO: 2 - RNA, 18 nt, artificial sequence
  inhibitory RNA
cguauaacaauaaggggc

SEQ ID NO: 3 - RNA, 19 nt, artificial sequence
  inhibitory RNA
ucguauaacaauaaggggc

SEQ ID NO: 4 - DNA, 19 bp, artificial sequence
  inhibitory RNA
tcguauaacaauaaggggc

SEQ ID NO: 5 - RNA, 18 nt, artificial sequence
  inhibitory RNA
ucguauaacaauaagggg

SEQ ID NO: 6 - RNA, 17 nt, artificial sequence
  inhibitory RNA
ucguauaacaauaaggg

SEQ ID NO: 7 - RNA, 17 nt, artificial sequence
  inhibitory RNA
uguauaacaauaagggg

SEQ ID NO: 8 - RNA, 19 nt, artificial sequence
  inhibitory RNA
ucguauaacaauaaggggc

SEQ ID NO: 9 - RNA, 19 nt, artificial sequence
  inhibitory RNA
gauaacucuguccauuacc

SEQ ID NO: 10 - RNA, 19 nt, artificial sequence
  inhibitory RNA
auaacucuguccauuacca

SEQ ID NO: 11 - RNA, 19 nt, artificial sequence
  inhibitory RNA
uaacucuguccauuaccgu

SEQ ID NO: 12 - RNA, 19 nt, artificial sequence
  inhibitory RNA
agaaugugccucgauaacu

SEQ ID NO: 13 - RNA, 19 nt, artificial sequence
  inhibitory RNA
auaacucuguccaucacca

SEQ ID NO: 14 - RNA, 19 nt, artificial sequence
  inhibitory RNA
auaacucuguccaucaccu

SEQ ID NO: 15 - RNA, 19 nt, artificial sequence
  inhibitory RNA
uaacucuguccauuaccau

SEQ ID NO: 16 - RNA, 19 nt, artificial sequence
  inhibitory RNA
augugccuugauaacucug

SEQ ID NO: 17 - RNA, 19 nt, artificial sequence
  inhibitory RNA
aguuggugcugcuucagaa

SEQ ID NO: 18 - RNA, 19 nt, artificial sequence
  inhibitory RNA
aauaaggggcugccacagg

SEQ ID NO: 19 - RNA, 19 nt, artificial sequence
  inhibitory RNA
uaacucuguccaucaccau

SEQ ID NO: 20 - RNA, 19 nt, artificial sequence
  inhibitory RNA
augagccucgauaacucug

SEQ ID NO: 21 - RNA, 19 nt, artificial sequence
  inhibitory RNA
aaugagccucgauaacucu

SEQ ID NO: 22 - RNA, 19 nt, artificial sequence
  inhibitory RNA
aaugcuuccaggacauuuc

SEQ ID NO: 23 - RNA, 19 nt, artificial sequence
  inhibitory RNA
acagugguggagaaugugc

SEQ ID NO: 24 - RNA, 19 nt, artificial sequence
  inhibitory RNA
guaugugccucgauaacuc

SEQ ID NO: 25 - RNA, 19 nt, artificial sequence
  inhibitory RNA
ucgauaacucuguccauca

SEQ ID NO: 26 - RNA, 19 nt, artificial sequence
  inhibitory RNA
ugucacuggacauuguguc

SEQ ID NO: 27 - RNA, 19 nt, artificial sequence
  inhibitory RNA
cugggauccaugguguaac

SEQ ID NO: 28 - RNA, 19 nt, artificial sequence
  inhibitory RNA
agaugaccaagcuuggcag

SEQ ID NO: 29 - RNA, 19 nt, artificial sequence
  inhibitory RNA
uuaacucuguccauuaccg

SEQ ID NO: 30 - RNA, 19 nt, artificial sequence
  inhibitory RNA
uuaacucuguccauuaccc

SEQ ID NO: 31 - RNA, 19 nt, artificial sequence
  inhibitory RNA
uuaacucuguccauuaccu

SEQ ID NO: 32 - RNA, 19 nt, artificial sequence
  inhibitory RNA
auaacucuguccauuaccc

SEQ ID NO: 33 - RNA, 19 nt, artificial sequence
  inhibitory RNA
auaacucuguccauuaccu

SEQ ID NO: 34 - RNA, 19 nt, artificial sequence
  inhibitory RNA
uuaacucuguccauuacca

SEQ ID NO: 35 - RNA, 18 nt, artificial sequence
  passenger sequence
cguauaacaauaaggggc

SEQ ID NO: 36 - RNA, 19 nt, artificial sequence
  passenger sequence
ucguauaacaauaaggggc

SEQ ID NO: 37 - DNA, 19 bp, artificial sequence
  passenger sequence
tcguauaacaauaaggggc

SEQ ID NO: 38 - RNA, 18 nt, artificial sequence
  passenger sequence
ucguauaacaauaagggg

SEQ ID NO: 39 - RNA, 17 nt, artificial sequence
  passenger sequence
ucguauaacaauaaggg

SEQ ID NO: 40 - RNA, 17 nt, artificial sequence
  passenger sequence
uguauaacaauaagggg

SEQ ID NO: 41 - RNA, 19 nt, artificial sequence
  passenger sequence
auaacucuguccauuaccg

SEQ ID NO: 42 - RNA, 22 nt, artificial sequence
  passenger sequence
acagccccuuauuguuauacga

SEQ ID NO: 43 - RNA, 18 nt, artificial sequence
  passenger sequence
gccccuuauuguuauacg

SEQ ID NO: 44 - RNA, 19 nt, artificial sequence
  passenger sequence
gccccuuauuguuauacga

SEQ ID NO: 45 - RNA, 19 nt, artificial sequence
  passenger sequence
gccccuuauuguuauacga

SEQ ID NO: 46 - RNA, 18 nt, artificial sequence
  passenger sequence
ccccuuauuguuauacga

SEQ ID NO: 47 - RNA, 17 nt, artificial sequence
  passenger sequence
cccuuauuguuauacga

SEQ ID NO: 48 - RNA, 17 nt, artificial sequence
  passenger sequence
ccccuuauuguuauaca

SEQ ID NO: 49 - RNA, 19 nt, artificial sequence
  passenger sequence
cgguaauggacagaguuau

SEQ ID NO: 50 - DNA, 40 bp, artificial sequence
  inhibitory RNA
  misc_feature (20)..(20): n is a, c, g, t or u
acgggacaguauucucagunatcacctcatcccgcgaagc

SEQ ID NO: 51 - DNA, 40 bp, artificial sequence
  inhibitory RNA
cccaauaaagcuggacaagaatcacctcatcccgcgaagc

SEQ ID NO: 52 - DNA, 40 bp, artificial sequence
  inhibitory RNA
cuguagguugcuuaaaagggatcacctcatcccgcgaagc

SEQ ID NO: 53 - DNA, 40 bp, artificial sequence
  inhibitory RNA
cuggagcaccguuaaggacaatcacctcatcccgcgaagc

SEQ ID NO: 54 - DNA, 40 bp, artificial sequence
  inhibitory RNA
ucccaauaaagcuggacaagatcacctcatcccgcgaagc

SEQ ID NO: 55 - DNA, 40 bp, artificial sequence
  inhibitory RNA
gccccuguagguugcuuaaaatcacctcatcccgcgaagc

SEQ ID NO: 56 - DNA, 40 bp, artificial sequence
  inhibitory RNA
cccugaaagacuacuggagcatcacctcatcccgcgaagc

SEQ ID NO: 57 - DNA, 40 bp, artificial sequence
  inhibitory RNA
ugcuuaaaagggacaguauuctcacctcatcccgcgaagc

SEQ ID NO: 58 - DNA, 40 bp, artificial sequence
  inhibitory RNA
gaccucaauaccccaaguccatcacctcatcccgcgaagc

SEQ ID NO: 59 - DNA, 40 bp, artificial sequence
  inhibitory RNA
gagcaccguuaaggacaaguutcacctcatcccgcgaagc

SEQ ID NO: 60 - RNA, 21 nt, artificial sequence
  inhibitory RNA
  misc_feature (20)..(20): n is a, c, g, t or u
acgggacaguauucucaguna

SEQ ID NO: 61 - RNA, 21 nt, artificial sequence
  inhibitory RNA
cccaauaaagcuggacaagaa

SEQ ID NO: 62 - RNA, 21 nt, artificial sequence
  inhibitory RNA
cuguagguugcuuaaaaggga

SEQ ID NO: 63 - RNA, 21 nt, artificial sequence
  inhibitory RNA
cuggagcaccguuaaggacaa

SEQ ID NO: 64 - RNA, 21 nt, artificial sequence
  inhibitory RNA
ucccaauaaagcuggacaaga

SEQ ID NO: 65 - RNA, 21 nt, artificial sequence
  inhibitory RNA
gccccuguagguugcuuaaaa

SEQ ID NO: 66 - RNA, 21 nt, artificial sequence
  inhibitory RNA
cccugaaagacuacuggagca

SEQ ID NO: 67 - RNA, 21 nt, artificial sequence
  inhibitory RNA
ugcuuaaaagggacaguauuc

SEQ ID NO: 68 - RNA, 21 nt, artificial sequence
  inhibitory RNA
gaccucaauaccccaagucca

SEQ ID NO: 69 - DNA, 22 bp, artificial sequence
  inhibitory RNA
gagcaccguuaaggacaaguut

SEQ ID NO: 70 - RNA, 21 nt, artificial sequence
  inhibitory RNA
ucacugagaauacugucccgu

SEQ ID NO: 71 - RNA, 21 nt, artificial sequence
  inhibitory RNA
uucuuguccagcuuuauuggg

SEQ ID NO: 72 - RNA, 21 nt, artificial sequence
  inhibitory RNA
ucccuuuuaagcaaccuacag

SEQ ID NO: 73 - RNA, 21 nt, artificial sequence
  inhibitory RNA
uuguccuuaacggugcuccag

SEQ ID NO: 74 - RNA, 21 nt, artificial sequence
  inhibitory RNA
ucuuguccagcuuuauuggga

SEQ ID NO: 75 - RNA, 21 nt, artificial sequence
  inhibitory RNA
uuuuaagcaaccuacaggggc

SEQ ID NO: 76 - RNA, 21 nt, artificial sequence
  inhibitory RNA
ugcuccaguagucuuucaggg

SEQ ID NO: 77 - RNA, 21 nt, artificial sequence
  inhibitory RNA
gaauacugucccuuuuaagca

SEQ ID NO: 78 - RNA, 21 nt, artificial sequence
  inhibitory RNA
uggacuugggguauugagguc

SEQ ID NO: 79 - RNA, 21 nt, artificial sequence
  inhibitory RNA
aacuuguccuuaacggugcuc

SEQ ID NO: 80 - DNA, 40 bp, artificial sequence
  inhibitory RNA
ucacugagaauacugucccgutcacctcatcccgcgaagc

SEQ ID NO: 81 - DNA, 40 bp, artificial sequence
  inhibitory RNA
uucuuguccagcuuuauugggtcacctcatcccgcgaagc

SEQ ID NO: 82 - DNA, 40 bp, artificial sequence
  inhibitory RNA
ucccuuuuaagcaaccuacagtcacctcatcccgcgaagc

SEQ ID NO: 83 - DNA, 40 bp, artificial sequence
  inhibitory RNA
uuguccuuaacggugcuccagtcacctcatcccgcgaagc

SEQ ID NO: 84 - DNA, 40 bp, artificial sequence
  inhibitory RNA
ucuuguccagcuuuauugggatcacctcatcccgcgaagc

SEQ ID NO: 85 - DNA, 40 bp, artificial sequence
  inhibitory RNA
uuuuaagcaaccuacaggggctcacctcatcccgcgaagc

SEQ ID NO: 86 - DNA, 40 bp, artificial sequence
  inhibitory RNA
ugcuccaguagucuuucagggtcacctcatcccgcgaagc

SEQ ID NO: 87 - DNA, 40 bp, artificial sequence
  inhibitory RNA
gaauacugucccuuuuaagcatcacctcatcccgcgaagc

SEQ ID NO: 88 - DNA, 40 bp, artificial sequence
  inhibitory RNA
uggacuugggguauugagguctcacctcatcccgcgaagc

SEQ ID NO: 89 - DNA, 40 bp, artificial sequence
  inhibitory RNA
aacuuguccuuaacggugcuctcacctcatcccgcgaagc

SEQ ID NO: 90 - DNA, 40 bp, artificial sequence
  inhibitory RNA
cucuguaaauuuggaaguguctcacctcatcccgcgaagc

SEQ ID NO: 91 - DNA, 40 bp, artificial sequence
  inhibitory RNA
caccaugagcuagguggaguatcacctcatcccgcgaagc

SEQ ID NO: 92 - DNA, 40 bp, artificial sequence
  inhibitory RNA
uuccugaagugacaaaggaaatcacctcatcccgcgaagc

SEQ ID NO: 93 - DNA, 40 bp, artificial sequence
  inhibitory RNA
gaccaccaggaacuauaucuutcacctcatcccgcgaagc

SEQ ID NO: 94 - DNA, 40 bp, artificial sequence
  inhibitory RNA
guuccagaaauacauugguuutcacctcatcccgcgaagc

SEQ ID NO: 95 - DNA, 40 bp, artificial sequence
  inhibitory RNA
aaccgcaagggcuuugugaaatcacctcatcccgcgaagc

SEQ ID NO: 96 - DNA, 40 bp, artificial sequence
  inhibitory RNA
gagcaagaaguucccaggcautcacctcatcccgcgaagc

SEQ ID NO: 97 - DNA, 40 bp, artificial sequence
  inhibitory RNA
caguaguaggcaucuggaaugtcacctcatcccgcgaagc

SEQ ID NO: 98 - DNA, 40 bp, artificial sequence
  inhibitory RNA
gucaugggugucuguggguuatcacctcatcccgcgaagc

SEQ ID NO: 99 - DNA, 40 bp, artificial sequence
  inhibitory RNA
gcucuguaaauuuggaagugutcacctcatcccgcgaagc

SEQ ID NO: 100 - RNA, 21 nt, artificial sequence
  inhibitory RNA
cucuguaaauuuggaaguguc

SEQ ID NO: 101 - RNA, 21 nt, artificial sequence
  inhibitory RNA
caccaugagcuagguggagua

SEQ ID NO: 102 - RNA, 21 nt, artificial sequence
  inhibitory RNA
uuccugaagugacaaaggaaa

SEQ ID NO: 103 - RNA, 21 nt, artificial sequence
  inhibitory RNA
gaccaccaggaacuauaucuu

SEQ ID NO: 104 - RNA, 21 nt, artificial sequence
  inhibitory RNA
guuccagaaauacauugguuu

SEQ ID NO: 105 - RNA, 21 nt, artificial sequence
  inhibitory RNA
aaccgcaagggcuuugugaaa

SEQ ID NO: 106 - RNA, 21 nt, artificial sequence
  inhibitory RNA
gagcaagaaguucccaggcau

SEQ ID NO: 107 - RNA, 21 nt, artificial sequence
  inhibitory RNA
caguaguaggcaucuggaaug

SEQ ID NO: 108 - RNA, 21 nt, artificial sequence
  inhibitory RNA
gucaugggugucuguggguua

SEQ ID NO: 109 - RNA, 21 nt, artificial sequence
  inhibitory RNA
gcucuguaaauuuggaagugu

SEQ ID NO: 110 - RNA, 21 nt, artificial sequence
  inhibitory RNA
gacacuuccaaauuuacagag

SEQ ID NO: 111 - RNA, 21 nt, artificial sequence
  inhibitory RNA
uacuccaccuagcucauggug

SEQ ID NO: 112 - RNA, 21 nt, artificial sequence
  inhibitory RNA
uuuccuuugucacuucaggaa

SEQ ID NO: 113 - RNA, 21 nt, artificial sequence
  inhibitory RNA
aagauauaguuccuggugguc

SEQ ID NO: 114 - RNA, 21 nt, artificial sequence
  inhibitory RNA
aaaccaauguauuucuggaac

SEQ ID NO: 115 - RNA, 21 nt, artificial sequence
  inhibitory RNA
uuucacaaagcccuugcgguu

SEQ ID NO: 116 - RNA, 21 nt, artificial sequence
  inhibitory RNA
augccugggaacuucuugcuc

SEQ ID NO: 117 - RNA, 21 nt, artificial sequence
  inhibitory RNA
cauuccagaugccuacuacug

SEQ ID NO: 118 - RNA, 21 nt, artificial sequence
  inhibitory RNA
uaacccacagacacccaugac

SEQ ID NO: 119 - RNA, 21 nt, artificial sequence
  inhibitory RNA
acacuuccaaauuuacagagc

SEQ ID NO: 120 - DNA, 40 bp, artificial sequence
  inhibitory RNA
gacacuuccaaauuuacagagtcacctcatcccgcgaagc

SEQ ID NO: 121 - DNA, 40 bp, artificial sequence
  inhibitory RNA
uacuccaccuagcucauggugtcacctcatcccgcgaagc

SEQ ID NO: 122 - DNA, 40 bp, artificial sequence
  inhibitory RNA
uuuccuuugucacuucaggaatcacctcatcccgcgaagc

SEQ ID NO: 123 - DNA, 40 bp, artificial sequence
  inhibitory RNA
aagauauaguuccuggugguctcacctcatcccgcgaagc

SEQ ID NO: 124 - DNA, 40 bp, artificial sequence
  inhibitory RNA
aaaccaauguauuucuggaactcacctcatcccgcgaagc

SEQ ID NO: 125 - DNA, 40 bp, artificial sequence
  inhibitory RNA
uuucacaaagcccuugcgguutcacctcatcccgcgaagc

SEQ ID NO: 126 - DNA, 40 bp, artificial sequence
  inhibitory RNA
augccugggaacuucuugcuctcacctcatcccgcgaagc

SEQ ID NO: 127 - DNA, 40 bp, artificial sequence
  inhibitory RNA
cauuccagaugccuacuacugtcacctcatcccgcgaagc

SEQ ID NO: 128 - DNA, 40 bp, artificial sequence
  inhibitory RNA
uaacccacagacacccaugactcacctcatcccgcgaagc

SEQ ID NO: 129 - DNA, 40 bp, artificial sequence
  inhibitory RNA
acacuuccaaauuuacagagctcacctcatcccgcgaagc

SEQ ID NO: 130 - DNA, 19 bp, artificial sequence
  single stranded DNA molecule
cgaagcgccctactccact

SEQ ID NO: 131 - RNA, 21 nt, artificial sequence
  inhibitory RNA
gaccaccaggaacuauaucuu

SEQ ID NO: 132 - RNA, 21 nt, artificial sequence
  inhibitory RNA
guuccagaaauacauugguuu

SEQ ID NO: 133 - RNA, 21 nt, artificial sequence
  inhibitory RNA
aaccgcaagggcuuugugaaa

SEQ ID NO: 134 - RNA, 21 nt, artificial sequence
  inhibitory RNA
gagcaagaaguucccaggcau

SEQ ID NO: 135 - RNA, 21 nt, artificial sequence
  inhibitory RNA
cuuuggagagaaugaagugua

SEQ ID NO: 136 - RNA, 21 nt, artificial sequence
  inhibitory RNA
cuucgacaagcacaagaccaa

SEQ ID NO: 137 - RNA, 21 nt, artificial sequence
  inhibitory RNA
gccgauggguccagaagaagu

SEQ ID NO: 138 - RNA, 21 nt, artificial sequence
  inhibitory RNA
cuucacuuggcugguguuuga

SEQ ID NO: 139 - RNA, 21 nt, artificial sequence
  inhibitory RNA
cuccuuuggagagaaugaagu

SEQ ID NO: 140 - RNA, 21 nt, artificial sequence
  inhibitory RNA
ugccauccucauguacauauu

SEQ ID NO: 141 - RNA, 21 nt, artificial sequence
  inhibitory RNA
ccgcaagggcuuugugaaacu

SEQ ID NO: 142 - RNA, 21 nt, artificial sequence
  inhibitory RNA
agcaagaaguucccaggcaua

SEQ ID NO: 143 - RNA, 21 nt, artificial sequence
  inhibitory RNA
aguguacaagcaggugaucuu

SEQ ID NO: 144 - RNA, 21 nt, artificial sequence
  inhibitory RNA
ugcugaccaccaggaacuaua

SEQ ID NO: 145 - RNA, 21 nt, artificial sequence
  inhibitory RNA
ccgauggguccagaagaaguu

SEQ ID NO: 146 - RNA, 21 nt, artificial sequence
  inhibitory RNA
uuuggagagaaugaaguguac

SEQ ID NO: 147 - RNA, 21 nt, artificial sequence
  inhibitory RNA
uggcgcuacuuucgagacuac

SEQ ID NO: 148 - RNA, 21 nt, artificial sequence
  inhibitory RNA
aaugccuguguugagggagua

SEQ ID NO: 149 - RNA, 21 nt, artificial sequence
  inhibitory RNA
aguuccagaaauacauugguu

SEQ ID NO: 150 - RNA, 21 nt, artificial sequence
  inhibitory RNA
cagaagugagcaagaaguucc

SEQ ID NO: 151 - RNA, 21 nt, artificial sequence
  inhibitory RNA
aagauauaguuccuggugguc

SEQ ID NO: 152 - RNA, 21 nt, artificial sequence
  inhibitory RNA
aaaccaauguauuucuggaac

SEQ ID NO: 153 - RNA, 21 nt, artificial sequence
  inhibitory RNA
uuucacaaagcccuugcgguu

SEQ ID NO: 154 - RNA, 21 nt, artificial sequence
  inhibitory RNA
augccugggaacuucuugcuc

SEQ ID NO: 155 - RNA, 21 nt, artificial sequence
  inhibitory RNA
uacacuucauucucuccaaag

SEQ ID NO: 156 - RNA, 21 nt, artificial sequence
  inhibitory RNA
uuggucuugugcuugucgaag

SEQ ID NO: 157 - RNA, 21 nt, artificial sequence
  inhibitory RNA
acuucuucuggacccaucggc

SEQ ID NO: 158 - RNA, 21 nt, artificial sequence
  inhibitory RNA
ucaaacaccagccaagugaag

SEQ ID NO: 159 - RNA, 21 nt, artificial sequence
  inhibitory RNA
acuucauucucuccaaaggag

SEQ ID NO: 160 - RNA, 21 nt, artificial sequence
  inhibitory RNA
aauauguacaugaggauggca

SEQ ID NO: 161 - RNA, 21 nt, artificial sequence
  inhibitory RNA
aguuucacaaagcccuugcgg

SEQ ID NO: 162 - RNA, 21 nt, artificial sequence
  inhibitory RNA
uaugccugggaacuucuugcu

SEQ ID NO: 163 - RNA, 21 nt, artificial sequence
  inhibitory RNA
aagaucaccugcuuguacacu

SEQ ID NO: 164 - RNA, 21 nt, artificial sequence
  inhibitory RNA
uauaguuccugguggucagca

SEQ ID NO: 165 - RNA, 21 nt, artificial sequence
  inhibitory RNA
aacuucuucuggacccaucgg

SEQ ID NO: 166 - RNA, 21 nt, artificial sequence
  inhibitory RNA
guacacuucauucucuccaaa

SEQ ID NO: 167 - RNA, 21 nt, artificial sequence
  inhibitory RNA
guagucucgaaaguagcgcca

SEQ ID NO: 168 - RNA, 21 nt, artificial sequence
  inhibitory RNA
uacucccucaacacaggcauu

SEQ ID NO: 169 - RNA, 21 nt, artificial sequence
  inhibitory RNA
aaccaauguauuucuggaacu

SEQ ID NO: 170 - RNA, 21 nt, artificial sequence
  inhibitory RNA
ggaacuucuugcucacuucug

SEQ ID NO: 171 - RNA, 21 nt, artificial sequence
  inhibitory RNA
ccucauaggccuggaguuuau

SEQ ID NO: 172 - RNA, 21 nt, artificial sequence
  inhibitory RNA
aggccuggaguuuauucggaa

SEQ ID NO: 173 - RNA, 21 nt, artificial sequence
  inhibitory RNA
cccucauaggccuggaguuua

SEQ ID NO: 174 - RNA, 21 nt, artificial sequence
  inhibitory RNA
aggucuggaaugcaaagucaa

SEQ ID NO: 175 - RNA, 21 nt, artificial sequence
  inhibitory RNA
ggccuggaguuuauucggaaa

SEQ ID NO: 176 - RNA, 21 nt, artificial sequence
  inhibitory RNA
caggucuggaaugcaaaguca

SEQ ID NO: 177 - RNA, 21 nt, artificial sequence
  inhibitory RNA
ccucaccaagauccugcaugu

SEQ ID NO: 178 - RNA, 21 nt, artificial sequence
  inhibitory RNA
acccucauaggccuggaguuu

SEQ ID NO: 179 - RNA, 21 nt, artificial sequence
  inhibitory RNA
caccagcauacagagugacca

SEQ ID NO: 180 - RNA, 21 nt, artificial sequence
  inhibitory RNA
aucuccuagacaccagcauac

SEQ ID NO: 181 - RNA, 21 nt, artificial sequence
  inhibitory RNA
uccuagacaccagcauacaga

SEQ ID NO: 182 - RNA, 21 nt, artificial sequence
  inhibitory RNA
cuggaguuuauucggaaaagc

SEQ ID NO: 183 - RNA, 21 nt, artificial sequence
  inhibitory RNA
gccuggaguuuauucggaaaa

SEQ ID NO: 184 - RNA, 21 nt, artificial sequence
  inhibitory RNA
gaggcagagacugauccacuu

SEQ ID NO: 185 - RNA, 21 nt, artificial sequence
  inhibitory RNA
uaggccuggaguuuauucgga

SEQ ID NO: 186 - RNA, 21 nt, artificial sequence
  inhibitory RNA
cacuucucugccaaagauguc

SEQ ID NO: 187 - RNA, 21 nt, artificial sequence
  inhibitory RNA
augcaaagucaaggagcaugg

SEQ ID NO: 188 - RNA, 21 nt, artificial sequence
  inhibitory RNA
ggucauggucaccgacuucga

SEQ ID NO: 189 - RNA, 21 nt, artificial sequence
  inhibitory RNA
ggcagcuguuuugcaggacug

SEQ ID NO: 190 - RNA, 21 nt, artificial sequence
  inhibitory RNA
gggcagguuggcagcuguuuu

SEQ ID NO: 191 - RNA, 21 nt, artificial sequence
  inhibitory RNA
auaaacuccaggccuaugagg

SEQ ID NO: 192 - RNA, 21 nt, artificial sequence
  inhibitory RNA
uuccgaauaaacuccaggccu

SEQ ID NO: 193 - RNA, 21 nt, artificial sequence
  inhibitory RNA
uaaacuccaggccuaugaggg

SEQ ID NO: 194 - RNA, 21 nt, artificial sequence
  inhibitory RNA
uugacuuugcauuccagaccu

SEQ ID NO: 195 - RNA, 21 nt, artificial sequence
  inhibitory RNA
uuuccgaauaaacuccaggcc

SEQ ID NO: 196 - RNA, 21 nt, artificial sequence
  inhibitory RNA
ugacuuugcauuccagaccug

SEQ ID NO: 197 - RNA, 21 nt, artificial sequence
  inhibitory RNA
acaugcaggaucuuggugagg

SEQ ID NO: 198 - RNA, 21 nt, artificial sequence
  inhibitory RNA
aaacuccaggccuaugagggu

SEQ ID NO: 199 - RNA, 21 nt, artificial sequence
  inhibitory RNA
uggucacucuguaugcuggug

SEQ ID NO: 200 - RNA, 21 nt, artificial sequence
  inhibitory RNA
guaugcuggugucuaggagau

SEQ ID NO: 201 - RNA, 21 nt, artificial sequence
  inhibitory RNA
ucuguaugcuggugucuagga

SEQ ID NO: 202 - RNA, 21 nt, artificial sequence
  inhibitory RNA
gcuuuuccgaauaaacuccag

SEQ ID NO: 203 - RNA, 21 nt, artificial sequence
  inhibitory RNA
uuuuccgaauaaacuccaggc

SEQ ID NO: 204 - RNA, 21 nt, artificial sequence
  inhibitory RNA
aaguggaucagucucugccuc

SEQ ID NO: 205 - RNA, 21 nt, artificial sequence
  inhibitory RNA
uccgaauaaacuccaggccua

SEQ ID NO: 206 - RNA, 21 nt, artificial sequence
  inhibitory RNA
gacaucuuuggcagagaagug

SEQ ID NO: 207 - RNA, 21 nt, artificial sequence
  inhibitory RNA
ccaugcuccuugacuuugcau

SEQ ID NO: 208 - RNA, 21 nt, artificial sequence
  inhibitory RNA
ucgaagucggugaccaugacc

SEQ ID NO: 209 - RNA, 21 nt, artificial sequence
  inhibitory RNA
caguccugcaaaacagcugcc

SEQ ID NO: 210 - RNA, 21 nt, artificial sequence
  inhibitory RNA
aaaacagcugccaaccugccc

SEQ ID NO: 211 - RNA, 21 nt, artificial sequence
  inhibitory RNA
cuggagcaccguuaaggacaa

SEQ ID NO: 212 - RNA, 21 nt, artificial sequence
  inhibitory RNA
cccugaaagacuacuggagca

SEQ ID NO: 213 - RNA, 21 nt, artificial sequence
  inhibitory RNA
gagcaccguuaaggacaaguu

SEQ ID NO: 214 - RNA, 21 nt, artificial sequence
  inhibitory RNA
acuggagcaccguuaaggaca

SEQ ID NO: 215 - RNA, 21 nt, artificial sequence
  inhibitory RNA
ccugaaagacuacuggagcac

SEQ ID NO: 216 - RNA, 21 nt, artificial sequence
  inhibitory RNA
aagacuacuggagcaccguua

SEQ ID NO: 217 - RNA, 21 nt, artificial sequence
  inhibitory RNA
caguucccugaaagacuacug

SEQ ID NO: 218 - RNA, 21 nt, artificial sequence
  inhibitory RNA
ggugaccgauggcuucaguuc

SEQ ID NO: 219 - RNA, 21 nt, artificial sequence
  inhibitory RNA
gggugaccgauggcuucaguu

SEQ ID NO: 220 - RNA, 21 nt, artificial sequence
  inhibitory RNA
acuacuggagcaccguuaagg

SEQ ID NO: 221 - RNA, 21 nt, artificial sequence
  inhibitory RNA
gacuacuggagcaccguuaag

SEQ ID NO: 222 - RNA, 21 nt, artificial sequence
  inhibitory RNA
uucaguucccugaaagacuac

SEQ ID NO: 223 - RNA, 21 nt, artificial sequence
  inhibitory RNA
guucccugaaagacuacugga

SEQ ID NO: 224 - RNA, 21 nt, artificial sequence
  inhibitory RNA
uggagcaccguuaaggacaag

SEQ ID NO: 225 - RNA, 21 nt, artificial sequence
  inhibitory RNA
cgccaccaagaccgccaagga

SEQ ID NO: 226 - RNA, 21 nt, artificial sequence
  inhibitory RNA
gggcugggugaccgauggcuu

SEQ ID NO: 227 - RNA, 21 nt, artificial sequence
  inhibitory RNA
gccaccaagaccgccaaggau

SEQ ID NO: 228 - RNA, 21 nt, artificial sequence
  inhibitory RNA
agacuacuggagcaccguuaa

SEQ ID NO: 229 - RNA, 21 nt, artificial sequence
  inhibitory RNA
ccaccaagaccgccaaggaug

SEQ ID NO: 230 - RNA, 21 nt, artificial sequence
  inhibitory RNA
ucccugaaagacuacuggagc

SEQ ID NO: 231 - RNA, 21 nt, artificial sequence
  inhibitory RNA
uuguccuuaacggugcuccag

SEQ ID NO: 232 - RNA, 21 nt, artificial sequence
  inhibitory RNA
ugcuccaguagucuuucaggg

SEQ ID NO: 233 - RNA, 21 nt, artificial sequence
  inhibitory RNA
aacuuguccuuaacggugcuc

SEQ ID NO: 234 - RNA, 21 nt, artificial sequence
  inhibitory RNA
uguccuuaacggugcuccagu

SEQ ID NO: 235 - RNA, 21 nt, artificial sequence
  inhibitory RNA
gugcuccaguagucuuucagg

SEQ ID NO: 236 - RNA, 21 nt, artificial sequence
  inhibitory RNA
uaacggugcuccaguagucuu

SEQ ID NO: 237 - RNA, 21 nt, artificial sequence
  inhibitory RNA
caguagucuuucagggaacug

SEQ ID NO: 238 - RNA, 21 nt, artificial sequence
  inhibitory RNA
gaacugaagccaucggucacc

SEQ ID NO: 239 - RNA, 21 nt, artificial sequence
  inhibitory RNA
aacugaagccaucggucaccc

SEQ ID NO: 240 - RNA, 21 nt, artificial sequence
  inhibitory RNA
ccuuaacggugcuccaguagu

SEQ ID NO: 241 - RNA, 21 nt, artificial sequence
  inhibitory RNA
cuuaacggugcuccaguaguc

SEQ ID NO: 242 - RNA, 21 nt, artificial sequence
  inhibitory RNA
guagucuuucagggaacugaa

SEQ ID NO: 243 - RNA, 21 nt, artificial sequence
  inhibitory RNA
uccaguagucuuucagggaac

SEQ ID NO: 244 - RNA, 21 nt, artificial sequence
  inhibitory RNA
cuuguccuuaacggugcucca

SEQ ID NO: 245 - RNA, 21 nt, artificial sequence
  inhibitory RNA
uccuuggcggucuugguggcg

SEQ ID NO: 246 - RNA, 21 nt, artificial sequence
  inhibitory RNA
aagccaucggucacccagccc

SEQ ID NO: 247 - RNA, 21 nt, artificial sequence
  inhibitory RNA
auccuuggcggucuugguggc

SEQ ID NO: 248 - RNA, 21 nt, artificial sequence
  inhibitory RNA
uuaacggugcuccaguagucu

SEQ ID NO: 249 - RNA, 21 nt, artificial sequence
  inhibitory RNA
cauccuuggcggucuuggugg

SEQ ID NO: 250 - RNA, 21 nt, artificial sequence
  inhibitory RNA
gcuccaguagucuuucaggga

SEQ ID NO: 251 - DNA, 19 bp, artificial sequence
  single stranded DNA
tcacctcatcccgcgaagc

SEQ ID NO: 252 - DNA, 23 bp, artificial sequence
  inhibitory RNA
ttuccggaccucaaauaagccuu

SEQ ID NO: 253 - DNA, 40 bp, artificial sequence
  inhibitory RNA
aggccuggaguuuauucggaatcacctcatcccgcgaagc

SEQ ID NO: 254 - DNA, 23 bp, artificial sequence
  inhibitory RNA
ttuccggaccucaaauaagccuu

(19) United States
(12) Patent Application Publication
(10) Pub. No.: US 2025/0304966 A1
(43) Pub. Date: Oct. 2, 2025

(54) TREATMENT OF CARDIOVASCULAR DISEASE

(71) Applicant: ARGONAUTE RNA LIMITED, Bristol (GB)

(72) Inventors: Michael Khan, Bristol (GB); Daniel Mitchell, Bristol (GB); Johnathan Matlock, Bristol (GB)

(73) Assignee: ARGONAUTE RNA LIMITED, Bristol (GB)

(21) Appl. No.: 18/268,773

(22) PCT Filed: Dec. 21, 2021
(86) PCT No.: PCT/EP2021/087142

§ 371 Date: Jun. 21, 2023

(30) Foreign Application Priority Data
Dec. 23, 2020 (GB) 2020534.0
Dec. 23, 2020 (GB) 2020554.8
Dec. 23, 2020 (GB) 2020561.3
Dec. 23, 2020 (GB) 2020562.1

(51) Int. Cl. C12N 15/113

(52) U.S. Cl. CPC C12N 15/1137; C12N 2310/14; C12N 2310/3515

(57) ABSTRACT

This disclosure relates to a nucleic acid comprising a double stranded RNA molecule comprising sense and antisense strands and further comprising a single stranded DNA molecule covalently linked to the 3′ end of either the sense or antisense RNA part of the molecule wherein the double stranded inhibitory RNA targets genes associated with cardiovascular disease in the treatment hypercholesterolemia and diseases associated with hypercholesterolemia such as cardiovascular disease.

### CROSS REFERENCE TO RELATED APPLICATIONS

This is the U.S. National Stage of International Application No. PCT/EP2021/087142, filed Dec. 21, 2021, which was published in English under PCT Article 21(2), which in turn claims the benefit of Great Britain Application No. 2020534.0, filed Dec. 23, 2020, Great Britain Application No. 2020554.8, filed Dec. 23, 2020, Great Britain Application No. 2020561.3, filed Dec. 23, 2020, and Great Britain Application No. 2020562.1, filed Dec. 23, 2020. The PCT application is herein incorporated by reference.

### INCORPORATION OF ELECTRONIC SEQUENCE LISTING

The electronic sequence listing, submitted herewith as an ASCII text file named 481 OPUS corrected_ST25 (48,474 bytes), created on Dec. 13, 2023, is herein incorporated by reference in its entirety.

### FIELD OF THE DISCLOSURE

This disclosure relates to a nucleic acid comprising a double stranded RNA molecule comprising sense and antisense strands and further comprising a single stranded DNA molecule covalently linked to the 3′ end of either the sense or antisense RNA part of the molecule wherein the double stranded inhibitory RNA targets of cardiovascular disease genes; pharmaceutical compositions comprising said nucleic acid molecule and methods for the treatment of diseases associated with increased levels of expression of cardiovascular disease genes, for example hypercholesterolemia.

### BACKGROUND TO THE DISCLOSURE

Cardiovascular disease associated with hypercholesterolemia, for example ischaemic cardiovascular disease, is a common condition and results in heart disease and a high incidence of death and morbidity and can be a consequence of poor diet, obesity, or an inherited dysfunctional gene. For example, high levels of lipoprotein (a) and other lipoproteins, is associated with atherosclerosis. Cholesterol is essential for membrane biogenesis in animal cells. The lack of water solubility means that cholesterol is transported around the body in association with lipoproteins. Apolipoproteins form together with phospholipids, cholesterol and lipids which facilitate the transport of lipids such as cholesterol, through the bloodstream to the different parts of the body. Lipoproteins are classified according to size and can form HDL (High-density lipoprotein), LDL (Low-density lipoprotein), IDL (intermediate-density lipoprotein), VLDL (very low-density lipoprotein) and ULDL (ultra-low-density lipoprotein) lipoproteins.

Lipoproteins change composition throughout their circulation comprising different ratios of apolipoproteins A (ApoA), B (ApoB), C (ApoC), D (ApoD) or E (ApoE), triglycerides, cholesterol and phospholipids. For example, ApoB is the main apolipoprotein of ULDL and LDL and has two isoforms apoB-48 and apoB-100. Both ApoB isoforms are encoded by one single gene and wherein the shorter ApoB-48 gene is produced after RNA editing of the ApoB-100 transcript at residue 2180 resulting in the creation of a stop codon. ApoB-100 is the main structural protein of LDL and serves as a ligand for a cell receptor which allows transport of, for example, cholesterol into a cell.

Familial hypercholesterolemia is an orphan disease and results from elevated levels of LDL cholesterol (LDL-C) in the blood. The disease is an autosomal dominant disorder with both the heterozygous (350-550 mg/dL LDL-C) and homozygous (650-1000 mg/dL LDL-C) states resulting in elevated LDL-C. The heterozygous form of familial hypercholesterolemia is around 1:500 of the population. The homozygous state is much rarer and is approximately 1:1,000,000. The normal levels of LDL-C are in the region 130 mg/dL.

Hypercholesterolemia is particularly acute in paediatric patients which if not diagnosed early can result in accelerated coronary heart disease and premature death. If diagnosed and treated early the child can have a normal life expectancy. In adults, high LDL-C, either because of mutation or other factors, is directly associated with increased risk of atherosclerosis which can lead to coronary artery disease, stroke or kidney disease. Lowering levels of LDL-C is known to reduce the risk of atherosclerosis and associated conditions. LDL-C levels can be lowered initially by administration of statins which block the de novo synthesis of cholesterol by inhibiting the HMG-CoA reductase. Some subjects can benefit from combination therapy which combines a statin with other therapeutic agents such as ezetimibe, colestipol or nicotinic acid. However, expression and synthesis of HMG-CoA reductase adapts in response to the statin inhibition and increases over time, thus the beneficial effects are only temporary or limited after statin resistance is established.

There is therefore a desire to identify alternative therapies that can be used alone or in combination with existing therapeutic approaches to control cardiovascular disease because of elevated LDL-C.

A technique to specifically ablate gene function is through the introduction of double stranded inhibitory RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically, but not exclusively, derived from exons of the gene which is to be ablated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

The inhibition of expression of lipoprotein (a) is known and the use of inhibitory RNA to silence expression of lipoprotein (a) is also known. For example, WO2019/092283 discloses the identification of specific siRNA sequences that target knock down of mRNA encoding lipoprotein (a) and their use in the treatment of cardiovascular diseases linked to elevated lipoprotein (a) expression such as coronary heart disease, aortic stenosis or stroke. Similarly, U.S. Pat. No. 9,932,586 discloses specific siRNA sequences that target lipoprotein (a) expression and their use in the treatment of cardiovascular diseases linked to elevated lipoprotein (a) expression such as Buerger's disease, coronary heart disease, renal artery stenosis, hyperapobetalipoproteinemia, cerebrovascular atherosclerosis, cerebrovascular disease, and venous thrombosis.

Over expression of APOC III is associated with atherosclerosis and type 2 diabetes. For example, WO2003/020765 discloses a vaccination approach to the control of atherosclerosis using immunogens derived from ApoCIII polypeptide and its use in controlling atherosclerotic plaques in coronary and cerebrovascular disease. A similar vaccination approach is disclosed 5 in WO2004/080375 and WO2001/064008. In WO2014/205449 and WO2014/179626 is disclosed the use of antisense oligonucleotides to improve insulin sensitivity and treat type II diabetes by targeting APOCIII expression.

Furthermore, WO2007/136989 and WO2005/019418 each disclose the use of antisense compounds directed to DGAT to regulate expression of DGAT2 and treat conditions that would benefit from reduction in DGAT2 expression in relation to conditions that would benefit from reduction in serum triglyceride levels such as hypercholesterolemia, cardiovascular disease, type II diabetes and metabolic syndrome. WO2018/093966 discloses the use of RNA silencing 10 directed to DGAT2 and diglyceride acyltransferase 1 (DGAT1) to treat obesity and obesity associated diseases such as hypercholesterolemia, cardiovascular disease, type II diabetes and metabolic syndrome. Similarly, WO2005/044981 discloses the use of siRNA to target DGAT2 amongst many other gene targets and their use in the treatment of diseases that would benefit from triglyceride regulation.

This disclosure relates to a nucleic acid molecule comprising a double stranded inhibitory RNA that is modified by the inclusion of a short DNA part linked to the 3′ end of either the sense or antisense inhibitory RNA and which forms a hairpin structure and is designed with reference to the nucleotide sequence encoding lipoprotein (a). U.S. Pat. No. 8,067,572, which is incorporated by reference in its entirety, discloses examples of said nucleic acid molecules. The double stranded inhibitory RNA uses solely or predominantly natural nucleotides and does not require modified nucleotides or sugars that prior art double stranded RNA molecules typically utilise to improve pharmacodynamics and pharmacokinetics. The disclosed double stranded inhibitory RNAs have activity in silencing cardiovascular gene targets with potentially fewer side effects.

### STATEMENTS OF THE INVENTION

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of a cardiovascular gene target associated with cardiovascular disease wherein said gene target is not apolipoprotein B (Apo B) and proprotein convertase subtilisin kexin type 9 (PCSK9) and wherein said single stranded DNA molecule comprises a nucleotide sequence that is adapted over at least part of its length to anneal by complementary base pairing to a part of said single stranded DNA to form a double stranded DNA structure wherein said double stranded inhibitory RNA consists of natural nucleotides.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of a cardiovascular gene target associated with cardiovascular disease wherein said gene target is not apolipoprotein B (Apo B) and proprotein convertase subtilisin kexin type 9 (PCSK9), or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

A “polymorphic sequence variant” is a sequence that varies by one, two, three or more nucleotides. We disclaim the content of priority applications GB1909500.9, GB1910526.1, GB2000906.4, GB2010004.6 and PCT/GB2020/051573 from the claimed subject matter of the current application. In each of the pending applications we disclose and claim nucleic acid molecules according to the invention designed with reference Apolipoprotein B. Also disclaimed is the content of GB patent application GB2003756.0, GB2010276.0 and PCT/EP2021/056540 and GB2103594.4 which discloses proprotein convertase subtilisin kexin type 9 (PCSK9) nucleic acid molecules according to the invention.

In a preferred embodiment of the invention wherein the 5′ end of said single stranded DNA molecule is covalently linked to the 3′ end of the sense strand of the double stranded inhibitory RNA molecule.

In a preferred embodiment of the invention wherein the 5′ end of said single stranded DNA molecule is covalently linked to the 3′ end of the antisense strand of the double stranded inhibitory RNA molecule.

In a preferred embodiment of the invention said loop portion comprises a region comprising the nucleotide sequence GNA or GNNA, wherein each N independently represents guanine (G), thymidine (T), adenine (A), or cytosine (C).

In a preferred embodiment of the invention said loop domain comprises G and C nucleotide bases.

In an alternative embodiment of the invention said loop domain comprises the nucleotide sequence GCGAAGC.

In a preferred embodiment of the invention said single stranded DNA molecule comprises the nucleotide sequence TCACCTCATCCCGCGAAGC (SEQ ID NO: 251).

In an alternative preferred embodiment of the invention said single stranded DNA molecule comprises the nucleotide sequence 5′ CGAAGCGCCCTACTCCACT 3′ (SEQ ID NO 130).

The inhibitory RNA molecules comprise or consist of natural nucleotide bases that do not require chemical modification. Moreover, in some embodiments of the invention, wherein the crook DNA molecule is linked to the 3′ end of the sense strand of said double stranded inhibitory RNA, the antisense strand is optionally provided with at least a two-nucleotide base overhang sequence. The two-nucleotide overhang sequence can correspond to nucleotides encoded by the target or are non-encoding. The two-nucleotide overhang can be two nucleotides of any sequence and in any order, for example UU, AA, UA, AU, GG, CC, GC, CG, UG, GU, UC, CU and dTdT.

In a preferred embodiment of the invention said inhibitory RNA molecule comprises a two-nucleotide overhang comprising or consisting of deoxythymidine dinucleotide (dTdT).

In a preferred embodiment of the invention said dTdT overhang is positioned at the 5′ end of said antisense strand.

In an alternative preferred embodiment of the invention said dTdT overhang is positioned at the 3′ end of said antisense strand.

In a preferred embodiment of the invention said dTdT overhang is positioned at the 5′ end of said sense strand.

In an alternative preferred embodiment of the invention said dTdT overhang is positioned at the 3′ end of said sense strand.

In a preferred embodiment of the invention said sense and/or said antisense strands comprises internucleotide phosphorothioate linkages.

In a preferred embodiment of the invention said sense strand comprises internucleotide phosphorothioate linkages.

Preferably, the 5′ and/or 3′ terminal two nucleotides of said sense strand comprises two internucleotide phosphorothioate linkage.

In a preferred embodiment of the invention said antisense strand comprises internucleotide phosphorothioate linkages.

Preferably, the 5′ and/or 3′ terminal two nucleotides of said antisense strand comprises two internucleotide phosphorothioate linkage.

In an alternative preferred embodiment of the invention said single stranded DNA molecule comprises one or more internucleotide phosphorothioate linkages.

In a preferred embodiment of the invention said nucleic acid molecule comprises a vinylphosphonate modification,

In a preferred embodiment of the invention said vinylphosphonate modification is to the 5′ terminal phosphate of said sense RNA strand.

In a preferred embodiment of the invention said vinylphosphonate modification is to the 5′ terminal phosphate of said antisense RNA strand.

In a preferred embodiment of the invention said double stranded inhibitory RNA molecule is between 10 and 40 nucleotides in length.

In a preferred embodiment of the invention said double stranded inhibitory RNA molecule is between 17 and 29 nucleotides in length.

In a preferred embodiment of the invention said double stranded inhibitory RNA molecule is 19 to 21 nucleotides in length. Preferably, 19 nucleotides in length.

In a preferred embodiment of the invention said cardiovascular gene target is Human Lipoprotein (a).

In an alternative embodiment of the invention said double stranded inhibitory RNA molecule comprises an antisense nucleotide sequence selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34.

In a preferred embodiment of the invention said double stranded inhibitory RNA molecule comprises an antisense nucleotide sequence comprising SEQ ID NO: 41 and a sense nucleotide sequence comprising SEQ ID NO: 49, wherein said single stranded DNA molecule is covalently linked to the 3′ end of the sense strand of the double stranded inhibitory RNA molecule.

In a preferred embodiment of the invention said double stranded inhibitory RNA molecule comprises an antisense nucleotide sequence comprising SEQ ID NO: 4 and a sense nucleotide sequence comprising SEQ ID NO: 44, wherein said single stranded DNA molecule is covalently linked to the 3′ end of the antisense strand of the double stranded inhibitory RNA molecule.

In a preferred embodiment of the invention said double stranded inhibitory RNA molecule comprises an antisense nucleotide sequence comprising SEQ ID NO: 5 and a sense nucleotide sequence comprising SEQ ID NO: 46, wherein said single stranded DNA molecule is covalently linked to the 3′ end of the antisense strand of the double stranded inhibitory RNA molecule.

In an alternative preferred embodiment of the invention said cardiovascular gene target is Human Apolipoprotein C III (Apo C III).

Preferably, said nucleic acid molecule comprises an RNA strand comprising a nucleotide sequence selected from the group consisting of: SEQ ID NO: 60, 61, 62, 63, 64, 65, 66, 67, 68, 69, 70, 71, 72, 73, 74, 75, 76, 77, 78 and 79.

In a preferred embodiment of the invention said nucleic acid molecule comprises an RNA strand comprising a nucleotide sequence selected from the group consisting of: SEQ ID NO: 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 and 250.

Preferably said nucleic acid molecule comprises an RNA strand comprising a nucleotide sequence selected from the group consisting of: SEQ ID NO: 50, 51, 52, 53, 54, 55, 56, 57, 58, 80, 81, 82, 83, 84, 85, 86, 87, 88 and 89.

In an alternative preferred embodiment of the invention said cardiovascular gene target is Human diglyceride acyltransferase 2 (DGAT2).

Preferably, said nucleic acid comprises an RNA strand comprising a nucleotide sequence selected from the group consisting of: 100, 101, 102, 103, 104, 105, 106, 107, 108, 109, 110, 111, 112, 113, 114, 115, 116, 117, 118 and 119.

Preferably, said nucleic acid comprises an RNA strand comprising a nucleotide sequence selected from the group consisting of: 131, 132, 133, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169 and 170.

Preferably said nucleic acid comprises an RNA strand comprising a nucleotide sequence selected from the group consisting of: SEQ ID NO: 90, 91, 92, 93, 94, 95, 96, 97, 98, 99, 120, 121, 122, 123, 124, 125, 126, 127, 128 and 129.

In a preferred embodiment of the invention said nucleic acid molecule is covalently linked to N-acetylgalactosamine.

In a further embodiment of the invention N-acetylgalactosamine is linked to either the antisense part of said inhibitory RNA or the sense part of said inhibitory RNA.

Preferably, N-acetylgalactosamine is linked to the 5′ terminus is of said sense RNA.

In an alternative embodiment of the invention N-acetylgalactosamine is linked to the 3′ terminus of said sense RNA.

In an alternative preferred embodiment of the invention said N-acetylgalactosamine is linked to the 3′ terminus of said antisense RNA.

In a preferred embodiment of the invention N-acetylgalactosamine is monovalent.

In a preferred embodiment of the invention N-acetylgalactosamine is divalent.

In an alternative embodiment of the invention N-acetylgalactosamine is trivalent.

In a preferred embodiment of the invention said nucleic acid molecule is covalently linked to a molecule comprising the structure:

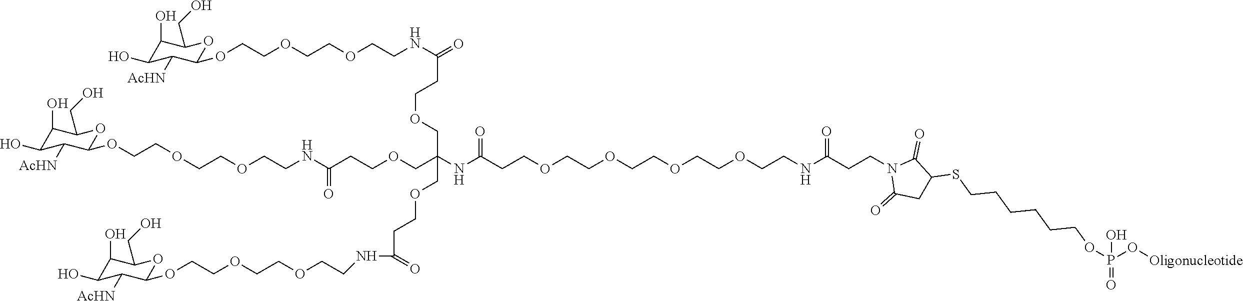

In an alternative embodiment of the invention said nucleic acid molecule is covalently linked to a molecule comprising the structure:

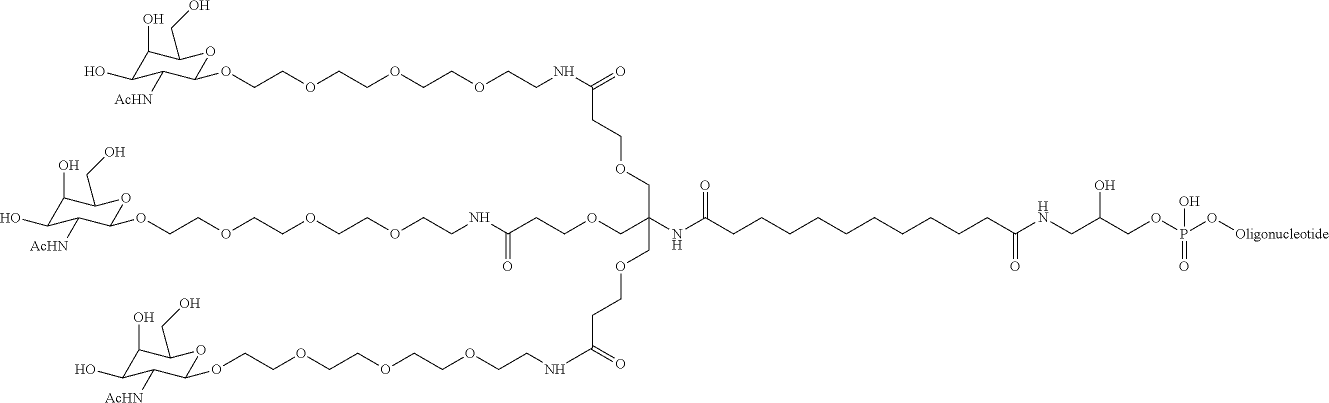

In an alternative embodiment of the invention said nucleic acid molecule is covalently linked to a molecule comprising the structure:

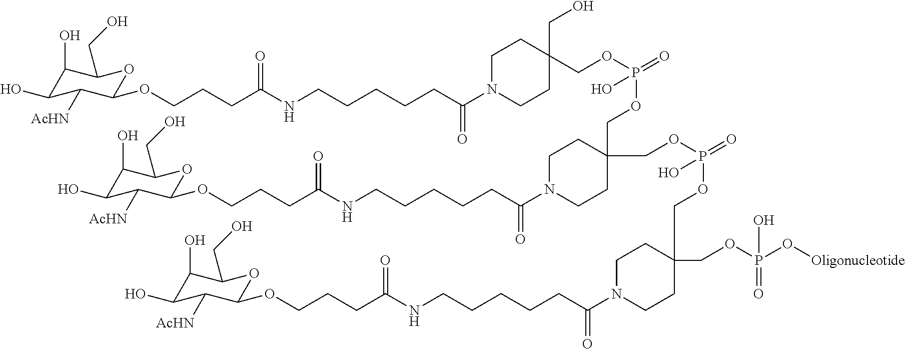

In an alternative embodiment of the invention said nucleic acid molecule is covalently linked to a molecule comprising the structure:

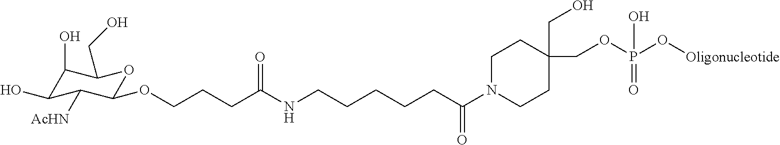

In an alternative preferred embodiment of the invention said nucleic acid molecule is covalently linked to a molecule comprising N-acetylgalactosamine 4-sulfate.

According to a further aspect of the invention there is provided a pharmaceutical composition comprising at least one nucleic acid molecule according to the invention.

In a preferred embodiment of the invention said composition further includes a pharmaceutical carrier and/or excipient.

When administered the compositions of the present invention are administered in pharmaceutically acceptable preparations. Such preparations may routinely contain pharmaceutically acceptable concentrations of salt, buffering agents, preservatives, compatible carriers and optionally other therapeutic agents, such as cholesterol lowering agents, which can be administered separately from the nucleic acid molecule according to the invention or in a combined preparation if a combination is compatible.

The combination of a nucleic acid according to the invention and the other, different therapeutic agent is administered as simultaneous, sequential or temporally separate dosages.

The therapeutics of the invention can be administered by any conventional route, including injection or by gradual infusion over time. The administration may, for example, be oral, intravenous, intraperitoneal, intramuscular, intracavity, subcutaneous, transdermal or transepithelial.

The compositions of the invention are administered in effective amounts. An “effective amount” is that amount of a composition that alone, or together with further doses, produces the desired response. In the case of treating a disease, such as cardiovascular disease, the desired response is inhibiting or reversing the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a nucleic acid molecule according to the invention for producing the desired response in a unit of weight or volume suitable for administration to a patient. The response can, for example, be measured by determining regression of cardiovascular disease and decrease of disease symptoms etc.

The doses of the nucleic acid molecule according to the invention administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. It will be apparent that the method of detection of the nucleic acid according to the invention facilitates the determination of an appropriate dosage for a subject in need of treatment.

In general, doses of the nucleic acid molecules herein disclosed of between 1 nM-1 μM generally will be formulated and administered according to standard procedures. Preferably doses can range from 1 nM-500 nM, 5 nM-200 nM, 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate or a transgenic mammal adapted for expression of human lipoprotein(a).

When administered, the pharmaceutical preparations of the invention are applied in pharmaceutically acceptable amounts and in pharmaceutically acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents e.g. statins. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Compositions may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “pharmaceutically acceptable carrier” in this context denotes an organic or inorganic ingredient, natural or synthetic, with which the active ingredient is combined to facilitate, for example, solubility and/or stability. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including acetic acid in a salt; citric acid in a salt; boric acid in a salt; and phosphoric acid in a salt. The pharmaceutical compositions also may contain, optionally, suitable preservatives.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Compositions suitable for oral administration may be presented as discrete units, such as capsules, tablets, lozenges, each containing a predetermined amount of the active compound.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

In a further preferred embodiment of the invention said pharmaceutical composition comprises at least one further, different, therapeutic agent.

In a preferred embodiment of the invention said further therapeutic agent is a statin.

Statins are commonly used to control cholesterol levels in subjects that have elevated LDL-C. Statins are effective in preventing and treating those subjects that are susceptible and those that have cardiovascular disease. The typical dosage of a statin is in the region 5 to 80 mg but this is dependent on the statin and the desired level of reduction of LDL-C required for the subject suffering from high LDL-C. However, expression and synthesis of HMG-CoA reductase, the target for statins, adapts in response to statin administration thus the beneficial effects of statin therapy are only temporary or limited after statin resistance is established.

Preferably said statin is selected from the group consisting of atorvastatin, fluvastatin, lovastatin, pitvastatin, pravastatin, rosuvastatin and simvastatin.

In a preferred embodiment of the invention said further therapeutic agent is ezetimibe. Optionally, ezetimibe is combined with at least one statin, for example simvastatin.

In an alternative preferred embodiment of the invention said further therapeutic agent is selected from the group consisting of fibrates, nicotinic acid, cholestyramine.

In a further alternative preferred embodiment of the invention said further therapeutic agent is a therapeutic antibody, for example, evolocumab, bococizumab or alirocumab.

According to a further aspect of the invention there is provided a nucleic acid molecule or a pharmaceutical composition according to the invention for use in the treatment or prevention of a subject that has or is predisposed to hypercholesterolemia or diseases associated with hypercholesterolemia.

In a preferred embodiment of the invention said subject is a paediatric subject.

A paediatric subject includes neonates (0-28 days old), infants (1-24 months old), young children (2-6 years old) and prepubescent [7-14 years old] children.

In an alternative preferred embodiment of the invention said subject is an adult subject.

In a preferred embodiment of the invention the hypercholesterolemia is familial hypercholesterolemia.

In a preferred embodiment of the invention familial hypercholesterolemia is associated with elevated levels of lipoprotein (a) expression.

In a preferred embodiment of the invention said subject is resistant to statin therapy.

In a preferred embodiment of the invention said disease associated with hypercholesterolemia is selected from the group consisting of: stroke prevention, hyperlipidaemia, cardiovascular disease, atherosclerosis, coronary heart disease, aortic stenosis, cerebrovascular disease, peripheral arterial disease, hypertension, metabolic syndrome, type II diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

According to a further aspect of the invention there is provided a method to treat a subject that has or is predisposed to hypercholesterolemia comprising administering an effective dose of a nucleic acid or a pharmaceutical composition according to the invention thereby treating or preventing hypercholesterolemia.

In a preferred method of the invention said subject is a paediatric subject.

In an alternative preferred method of the invention said subject is an adult subject.

In a preferred method of the invention the hypercholesterolemia is familial hypercholesterolemia.

In a preferred method of the invention familial hypercholesterolemia is associated with elevated levels of lipoprotein (a) expression.

In a preferred method of the invention said subject is resistant to statin therapy.

In a preferred method of the invention said disease associated with hypercholesterolemia is selected from the group consisting of: stroke prevention, hyperlipidaemia, cardiovascular disease, atherosclerosis, coronary heart disease, aortic stenosis, cerebrovascular disease, peripheral arterial disease, hypertension, metabolic syndrome, type II diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

According to a further aspect of the invention there is provided a treatment regimen for the diagnosis and treatment of hypercholesterolemia associated with elevated lipoprotein (a) comprising:
i) obtaining a biological sample from a subject suspected on having or suspected of having hypercholesterolemia;
ii) contacting the sample with an antibody, or antibody fragment, specific for an lipoprotein (a) polypeptide;
iii) determining the concentration lipoprotein (a) polypeptide and LDL-C in said biological sample; and
iv) administering a nucleic acid molecule or pharmaceutical composition according to the invention if the LDL-C concentration is greater than 350 mg/dL.

Typically, in familial hypercholesterolemia disease the levels of LDL-C are 350-550 mg/dL in subjects that are heterozygous for a selected mutation and 650-1000 mg/dL in those subjects carrying a homozygous mutation. The normal levels of LDL-C are in the region 130 mg/dL.

Throughout the description and claims of this specification, the words “comprise” and “contain” and variations of the words, for example “comprising” and “comprises”, means “including but not limited to” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures.

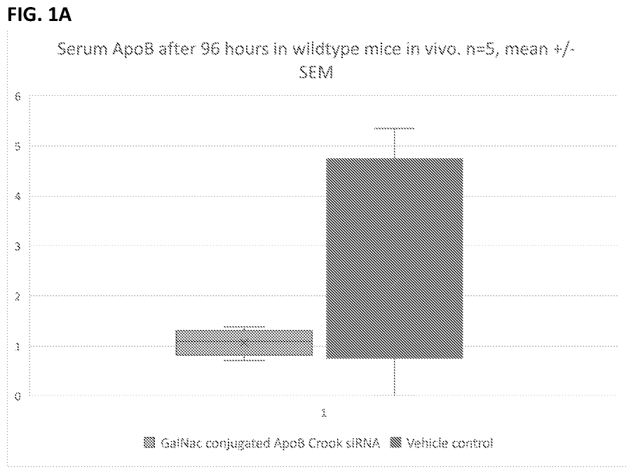

FIGS. 1A and 1B. Graphs illustrating in vivo activity of GalNAc-conjugated Crook anti-mouse ApoB siRNA compared to control siRNA constructs.

(FIG. 1A) Plasma ApoB levels (micrograms/ml) from five adult male wild-type C57BL/6 mice, were measured 96 hours following administration of GalNAc-conjugated ApoB Crook siRNA (one treatment group) and compared with the control treatment group administered with saline. Statistical analysis was applied using the two-tailed paired T test algorithm. Results show a substantive reduction in mean plasma ApoB levels in mice treated with GalNAc-conjugated Crook siRNA, compared to control. However, it just fails significance (p=0.11), most likely due to small sample size and variation in ApoB levels between control animals;

(FIG. 1B) Plasma ApoB levels (micrograms/ml) from five adult male wild-type C57BL/6 mice, were measured 96 hours following administration of GalNAc-conjugated ApoB Crook siRNA (one treatment group) and compared with the control treatment group, administered with siRNA construct unconjugated (without GalNAc) ApoB Crook siRNA. Statistical analysis was applied using the two-tailed paired T test algorithm. Results show a highly significant reduction in plasma ApoB levels in this GalNAc-conjugated Crook siRNA treatment group when compared to control unconjugated siRNA with Crook (P=0.00435832);

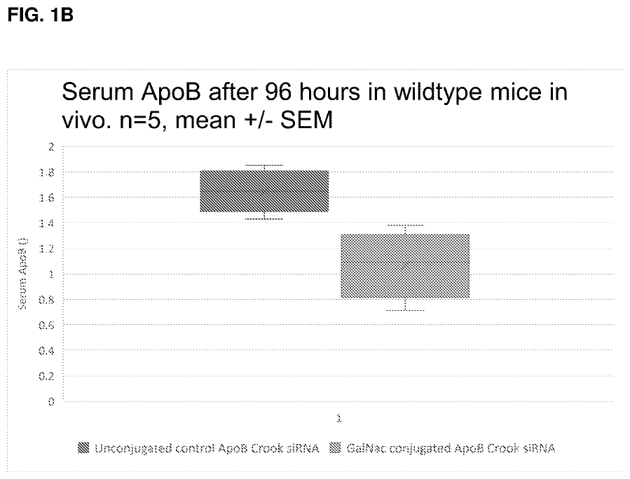

FIG. 2 Evaluation of transfection in RT4 cells using lethal siRNA reagents. 72 h post-transfection with indicated siRNAs, RT4 cell viability was quantified using the Cell Titer Glo 2.0 reagent (Promega—the luminescence value is proportional to the number of live cells). This bar graph shows the average luminescence (+SD, N=4);

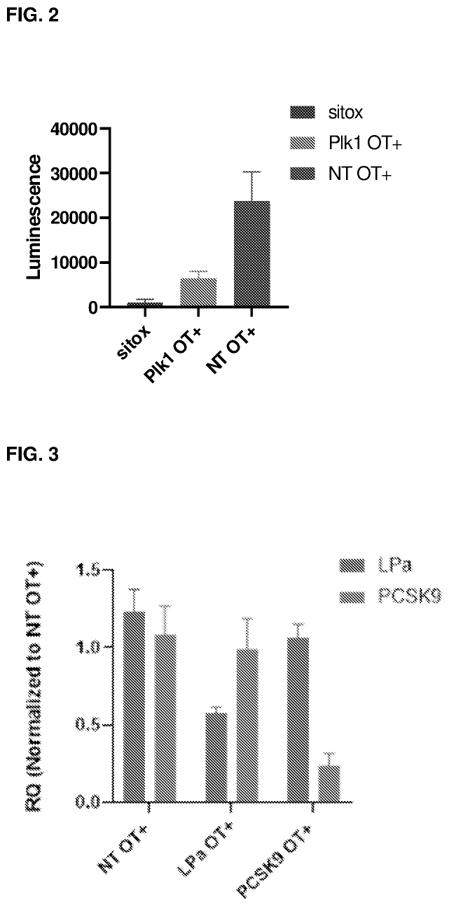

FIG. 3 Evaluation of transfection in RT4 cells using siRNA targeting LP(a) and PCSK9. 72 h post-transfection with indicated OT+ siRNAs, LP(a) (green bars) and PCSK9 (golden bars) mRNA was quantified by Duplex RT-qPCR. Represented data are Mean (+SEM) mRNA fold change normalized to NT OT+ (N=4);

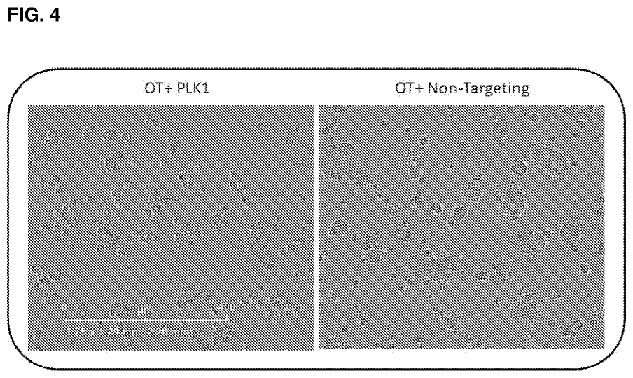

FIG. 4 Microscopy images showing RT4 cell death following OT+ PLK1 siRNA treatment. 72 h post transfection with the indicated siRNA at 25 nM, RT4 cells were imaged using an S3 IncuCyte instrument (Sartorius);

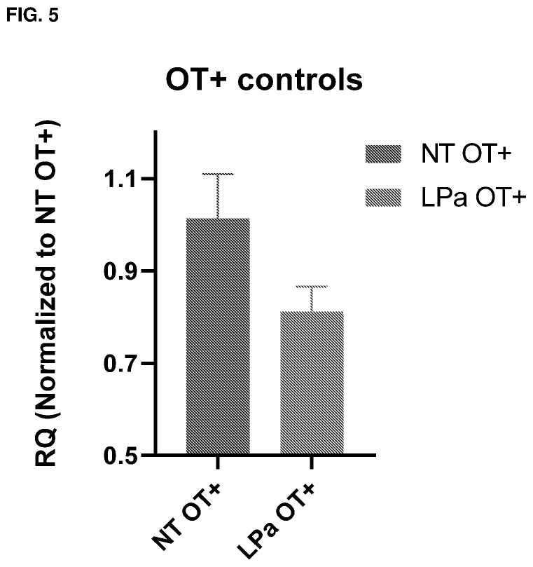

FIG. 5 Average LP(a) mRNA levels in cells treated with ON-TARGETplus Non-Targeting and LP(a) siRNAs at 25 nM. 72 h post-transfection, LP(a) mRNA level was quantified by duplex RT-qPCR. Represented data are Mean+SEM of LP(a) mRNA fold change relative to NT OT+(N=4);

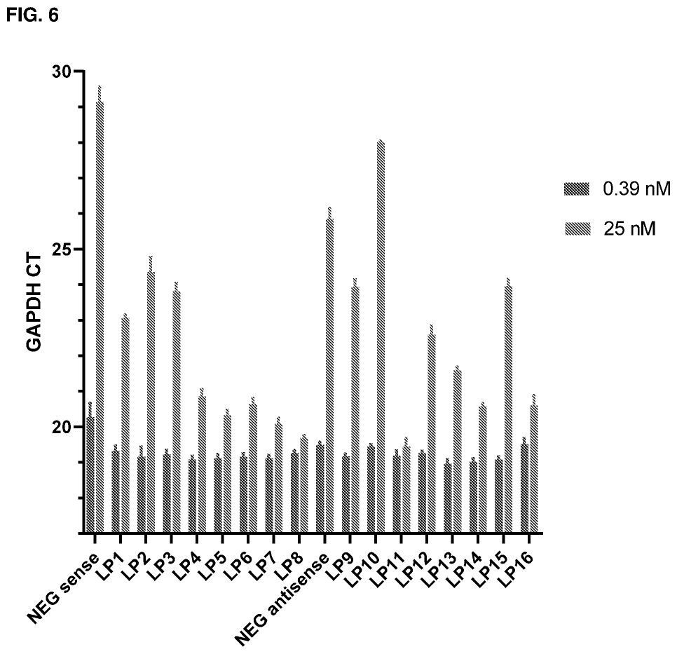

FIG. 6 GAPDH CT value following siRNA treatment. RT4 cells transfected with the indicated siRNAs at 0.39 nM (violet bar) or 25 nM (golden bar) were assessed for relative GAPDH mRNA level 72 h post transfection. Represented data are Mean+SEM of the GAPDH CT value (N=4);

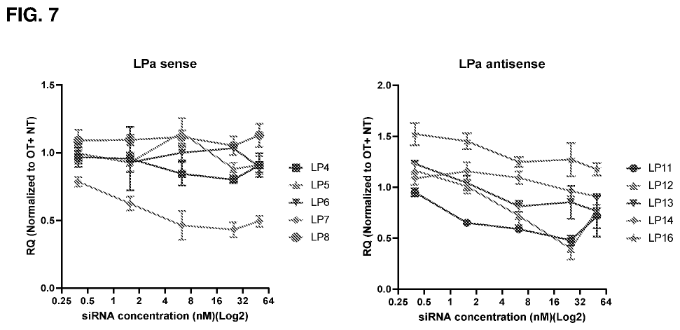

FIG. 7 RT4 were reverse transfected with five concentrations (0.39; 1.56; 6.25; 25 and 50 nM) of the indicated custom crook siRNAs. LP(a) mRNA levels relative to the OT+NT control was calculated for each data point. Represented are Mean+/−SEM (N=4);

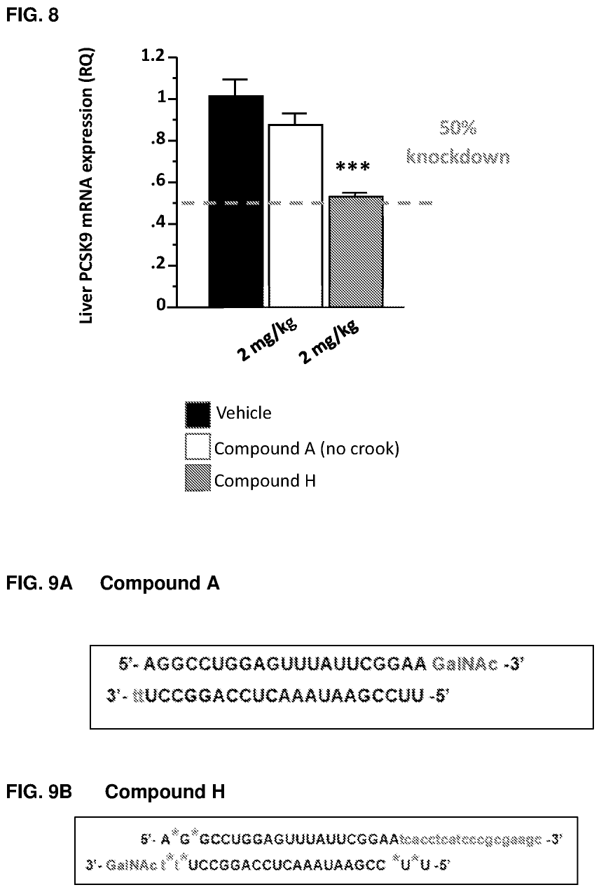

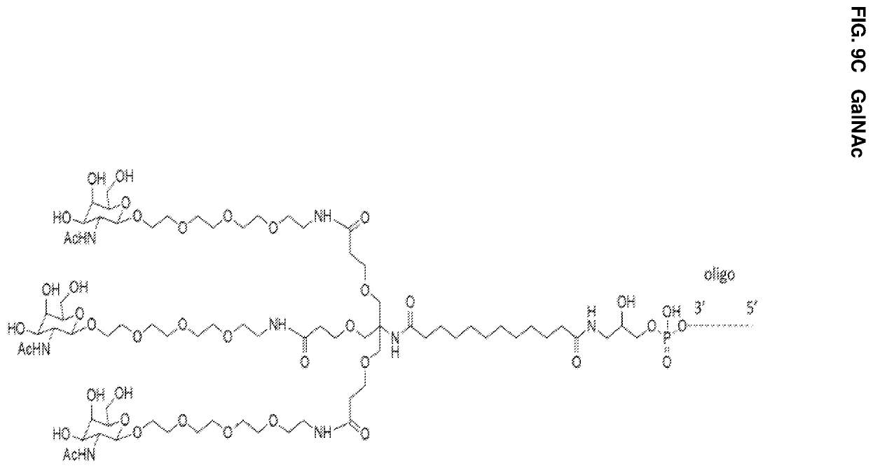

FIG. 8 An in vivo mouse study was performed to assess knockdown activity of GalNAc-conjugated Crook anti-mouse PCSK9 siRNA (Compound H; depicted in FIG. 9) compared to its ‘no Crook’ siRNA control (Compound A; depicted in FIG. 9). Compound H (at 2 mg/kg) shows significant knockdown of liver PCSK9 mRNA 48 hrs after SC injection, compared to Compound A and Vehicle control. Statistical analysis was applied using the two-tailed paired T test algorithm (p<0.001); and

FIGS. 9A-9C siRNA constructs administered in PCSK9 in vivo study (FIG. 8): (FIG. 9A) Compound A is a GalNAc-conjugated anti-mouse PCSK9 siRNA without a Crook moiety (SEQ ID NOs: 172 and 252); (FIG. 9B) Compound H is a GalNAc-conjugated PCSK9 siRNA with Crook attached to the 3′ of the sense strand (SEQ ID NOs: 253 and 254); (FIG. 9C) GalNAc structure. c, g, a, t: DNA bases; A, G, C, U: RNA bases; *internucleotide linkage phosphorothioate (PS).

### MATERIALS AND METHODS

### Lipoprotein (a)

Lipoprotein (a) is only expressed in the liver of human and non-human primate species, other species such as rodent species, do not have a gene that is equivalent to lipoprotein (a). However, apart from testing in non-human primates such as cynomolgus macaques, there are also several transgenic animal models adapted for the testing of agents effective at modulating lipoprotein (a). For example, U.S. Pat. No. 9,018,437 discloses a transgenic murine model for the expression of human lipoproteins including lipoprotein (a). U.S. Pat. No. 6,512,161 discloses a transgenic rabbit model for the expression of human lipoprotein (a); the contents of each disclosure are incorporated by reference in their entirety and provide in vivo models to test nucleic acid molecules according to the invention. Moreover, non-human primate models for testing therapeutic agents are well known. U.S. Pat. No. 9,932,586, the content of which is incorporated by reference in its entirety, including siRNA sequences that silence human lipoprotein (a), discloses the testing of siRNA agents in cynomolgus macaques and transgenic mouse models for expression of lipoprotein (a). WO2019/092283, the content of which is incorporated by reference in its entirety, including siRNA sequences that silence human lipoprotein (a), disclose the use of in vitro testing using primary human and non-human primate cell-lines. Dosages and administration routes for delivering apolipoprotein (a) crook nucleic acid is not appreciably different when administering to a non-human, non-human primate transgenic model as detailed above.

Rt4 Reverse Transfection (LP(a) siRNAs)
A description of the custom library evaluated in this study is provided in Table 1. Custom duplex siRNAs synthesized by Horizon Discovery were resuspended in UltraPure DNase and RNase free water to generate a stock solution of 10 μM.
Stock siRNAs were dispensed into 4×384-well assay plates:
   Plate 1: LP1-8 replicate 1
   Plate 2: LP1-8 replicate 2
   Plate 3: LP9-16 replicate 1
   Plate 4: LP9-16 replicate 2
On each assay plate, 8 Custom siRNAs and corresponding control (NEG sense or NEG antisense) were dispensed at the five concentrations (50 nM; 25 nM; 6.25 nM; 1.56 nM; 0.39 nM) in the assay plate. ON-TARGETplus controls consisting of siRNAs Non-Targeting (Horizon Discovery #D-001810-10-05), targeting PLK1 (Horizon Discovery #L-003290-00-0005) and targeting LP(a) (Horizon Discovery #L-020011-00-0005) were dispensed to give a final concentration of 25 nM.
Lipofectamine RNAiMAX (ThermoFisher #13778075) was diluted in OptiMEM media before 10 μL of the Lipfectamine RNAiMAX:OptiMEM solution was added per well to the assay plate. The final volume of RNAiMAX per well was 0.08 μL.
The lipid-siRNA mix was incubated 30 min at room temperature before adding the cells.
RT4 cells were diluted in assay media (McCoy's 5A GlutaMAX (GIBCO) 10% FBS 1% Pen/Strep) before 4,000 RT4 cells were seeded into each well of the assay plate in 40 μL volume. Quadruplicate technical replicates were seeded per assay condition.
The plates were incubated 72 h at 37° C., 5% CO₂ in a humidified atmosphere, prior to assessment of the cells.
Plates 1 and 3 were processed for Duplex RT-qPCR. Plates 2 and 4 were stored at −80° C. after media removal.
   ApoC3, DGAT2 and Lp(a) In Vitro Screen of Crook siRNA Activity in HepG2 Cells and RT4 Cells (Lp(a))

A description of the custom library siRNAs for ApoC3 and DGAT2 evaluated in this study is provided in Table 4.

### HepG2 and RT4 Reverse Transfection

Custom duplex siRNAs synthesized by Horizon Discovery (Lp(a)), and Bio-Synthesis (Lewisville, TX) for ApoC3 and DGAT2, were resuspended in UltraPure DNase and RNase free water to generate a stock solution of 10 μM.
Stock siRNAs were dispensed into 4×384-well assay plates (Greiner #781092 or Thermo Scientific™ 164688). On each assay plate, 10 Custom siRNAs for each target and 3 controls (POS lipoprotein (a), ApoB, NEG sense and NEG antisense) were dispensed to generate five-point four-fold dilution series from a top final concentration in the assay plate of 100 nM. ON TARGETplus Non-Targeting and lipoprotein (a) siRNAs controls were dispensed to give a final concentration of 25 nM. For ApoC3 and DGAT2, cells receiving no siRNA treatment were used as Negative controls.
Lipofectamine RNAiMAX (ThermoFisher) was diluted in Optimem media before 10 μL of the Lipofectamine RNAiMAX:OptiMEM solution was added per well to the assay plate. The final volume of RNAiMAX per well was 0.08 μL.
The lipid-siRNA mix was incubated 30 min at room temperature.
HepG2 cells were diluted in assay media (MEM GlutaMAX (GIBCO) 10% FBS 1% Pen/Strep) before 4,000 HepG2 cells were seeded into each well of the assay plate in 40 μL volume. Quadruplicate technical replicates were seeded per assay condition.
The plates were incubated 72 h at 37° C., 5% CO₂ in a humidified atmosphere, prior to assessment of the cells.

### Duplex RT-qPCR

72 h post-transfection, cells were processed for RT-qPCR read-out using the Cells-to-CT 1-step TaqMan Kit (Invitrogen 4391851C or A25603). Briefly, cells were washed with 50 μl ice-cold PBS and then lysed in 20 μl Lysis solution containing DNase I. After 5 min, lysis was stopped by addition of 2 μl STOP Solution for 2 min.
For the Lp(a) RT-qPCR analysis, 3 μl of lysate was dispensed per well into 384-well PCR plate as template in an 11 μl RT-qPCR reaction volume. For ApoC3 and DGAT2, 1 ul of lysate was dispensed per well into 96-well PCR plate as template in an 20 μl RT-qPCR reaction volume.
RT-qPCR for Lp(a) was performed using the ThermoFisher TaqMan Fast Virus1-Step Master Mix (#4444434) with TaqMan probes for GAPDH (VIC #4448486) and lipoprotein (a). For ApoC3 and DGAT2, RT-qPCR was performed using the TaqMan® 1-Step qRT-PCR Mix and Cells-to-CT 1-step TaqMan Kit, with TaqMan probes for GAPDH (VIC_PL, Assay Id Hs00266705_g1), ApoC3 (FAM, Assay Id Hs00163644_m1), and DGAT2 (FAM, Hs01045913_m1).
RT-qPCR was performed using a QuantStudio 5 or 6 thermocycling instrument (Applied BioSystems).
Relative quantification was determined using the ΔΔCT method, where GAPDH was used as internal control and expression changes normalized to the reference sample (either NEG sense or NEG antisense siRNA treated cells, or ‘no treatment’ cells).

### Statistics

For all assays in this project, four technical replicates were obtained for each data point.
Mean and Standard Error of the Mean (SEM) were calculated using Excel or Graphpad Prism.

All graphs were generated using Graphpad Prism.

### Crook Lipoprotein (a) or ApoB Constructs

Unconjugated and conjugated versions of lipoprotein(a) or ApoB Crook-siRNA were administered by IV and SC routes respectively to investigate the relative plasma and tissue exposure. The rationale for dose selection was based on the following information published in the scientific literature:

The GalNAc conjugated siRNA is dosed subcutaneously at 5 mg/kg which is expected to produce the required level of gene silencing where the ED₈₀ of structurally related siRNAs has been reported as 2.5 mg/kg (Soutschek et al., 2004). These structurally related siRNAs were tolerated up to 25 mg/kg, single administration, in the mouse (Soutschek et al., 2004).

The unconjugated version of siRNA is administered at 50 mg/kg intravenously. This 10-fold increase in the IV compared to the SC dose is due to the unconjugated siRNA being less effective at targeting the liver. Additionally, it is reported by Soutschek et al (2004) that lower levels of RNA are measured in the liver following IV compared to SC administration. It is stated that slower release of the siRNA from the subcutaneous depot leads to prolonged exposure increasing the potential for receptor-ligand interactions and greater uptake into the tissue. Similar related siRNA has been well tolerated by mice at up to 50 mg/kg IV administered on 3 consecutive days (Nair et al. 2014). As a precaution a 15 minute observation period is left between dosing the 1^{st} animal IV to determine if the test substance causes any adverse effects before the remaining animals are dosed.

The mouse or transgenic equivalent is the species of choice because it is used as one of the toxicology species in the safety testing of the test substance. There is a considerable amount of published data available which are acceptable to the regulatory authorities for assessing the significance to man of data generated in this species.

A library of duplex siRNAs (16 targeting LP(a)) was synthesized by Horizon Discovery. Table 1 shows the sequences of both strands of RNA for each siRNA. The following DNA sequence (TCACCTCATCCCGCGAAGC; SEQ ID NO: 251) was appended to the 3′ end of either the sense strand (siRNAs PC1 to PC10 and LP1 to LP8, thereafter referred to as sense siRNAs) or the antisense strand (siRNAs PC11 to PC20 and LP9 to LP16, thereafter referred to as antisense siRNAs).

TABLE 1
Selection of Lp(a) candidate siRNA sequences to which crook is conjugated

| siRNA ID | | | |
|---|---|---|---|
| Sense | Antisense | | |
| (SEQ ID | (SEQ ID | Sense sequence | Antisense sequence |
| NO) | NO) | (5′-3′) | (5′-3′) |
| LP1 (43) | LP9 (2) | GCCCCUUAUUGUUAUACG | CGUAUAACAAUAAGGGGC |
| LP2 (44) | LP10 (3) | GCCCCUUAUUGUUAUACGA | UCGUAUAACAAUAAGGGGC |
| LP3 (44) | LP11 (4) | GCCCCUUAUUGUUAUACGA | TCGUAUAACAAUAAGGGGC |
| LP4 (46) | LP12 (5) | CCCCUUAUUGUUAUACGA | UCGUAUAACAAUAAGGGG |
| LP5 (47) | LP13 (6) | CCCUUAUUGUUAUACGA | UCGUAUAACAAUAAGGG |
| LP6 (48) | LP14 (7) | CCCCUUAUUGUUAUACA | UGUAUAACAAUAAGGGG |
| LP7 (49) | LP15 (41) | CGGUAAUGGACAGAGUUAU | AUAACUCUGUCCAUUACCG |
| LP8 (42) | LP16 (35) | ACAGCCCCUUAUUGUUAUACGA | CGUAUAACAAUAAGGGGC |

TABLE 2
Selection of APOC III and DGAT 2 siRNA sequences to which crook is
conjugated

| SEQ ID | | |
|---|---|---|
| NO | Name | Sequence |
| 50 | APOC3_01 | 5′-ACGGGACAGUAUUCUCAGUNAtcacctcatcccgcgaagc-3′ |
| 51 | APOC3_02 | 5′-CCCAAUAAAGCUGGACAAGAAtcacctcatcccgcgaagc-3′ |
| 52 | APOC3_03 | 5′-CUGUAGGUUGCUUAAAAGGGAtcacctcatcccgcgaagc-3′ |
| 53 | APOC3_04 | 5′-CUGGAGCACCGUUAAGGACAAtcacctcatcccgcgaagc-3′ |
| 54 | APOC3_05 | 5′-UCCCAAUAAAGCUGGACAAGAtcacctcatcccgcgaagc-3′ |
| 55 | APOC3_06 | 5′-GCCCCUGUAGGUUGCUUAAAAtcacctcatcccgcgaagc-3′ |
| 56 | APOC3_07 | 5′-CCCUGAAAGACUACUGGAGCAtcacctcatcccgcgaagc-3′ |
| 57 | APOC3_08 | 5′-UGCUUAAAAGGGACAGUAUUCtcacctcatcccgcgaagc-3′ |
| 58 | APOC3_09 | 5′-GACCUCAAUACCCCAAGUCCAtcacctcatcccgcgaagc-3′ |
| 59 | APOC3_10 | 5′-GAGCACCGUUAAGGACAAGUUtcacctcatcccgcgaagc-3′ |
| 60 | APOC3_01 | 5′-ACGGGACAGUAUUCUCAGUNA |
| 61 | APOC3_02 | 5′-CCCAAUAAAGCUGGACAAGAA |
| 62 | APOC3_03 | 5′-CUGUAGGUUGCUUAAAAGGGA |
| 63 | APOC3_04 | 5′-CUGGAGCACCGUUAAGGACAA |
| 64 | APOC3_05 | 5′-UCCCAAUAAAGCUGGACAAGA |
| 65 | APOC3_06 | 5′-GCCCCUGUAGGUUGCUUAAAA |
| 66 | APOC3_07 | 5′-CCCUGAAAGACUACUGGAGCA |
| 67 | APOC3_08 | 5′-UGCUUAAAAGGGACAGUAUUC |
| 68 | APOC3_09 | 5′-GACCUCAAUACCCCAAGUCCA |
| 69 | APOC3_10 | 5′-GAGCACCGUUAAGGACAAGUUt |
| 70 | APOC3_01 | 5′-UCACUGAGAAUACUGUCCCGU-3′ |
| 71 | APOC3_02 | 5′-UUCUUGUCCAGCUUUAUUGGG-3′ |
| 72 | APOC3_03 | 5′-UCCCUUUUAAGCAACCUACAG-3′ |
| 73 | APOC3_04 | 5′-UUGUCCUUAACGGUGCUCCAG-3′ |
| 74 | APOC3_05 | 5′-UCUUGUCCAGCUUUAUUGGGA-3′ |
| 75 | APOC3_06 | 5′-UUUUAAGCAACCUACAGGGGC-3′ |
| 76 | APOC3_07 | 5′-UGCUCCAGUAGUCUUUCAGGG-3′ |
| 77 | APOC3_08 | 5′-GAAUACUGUCCCUUUUAAGCA-3′ |
| 78 | APOC3_09 | 5′-UGGACUUGGGGUAUUGAGGUC-3′ |
| 79 | APOC3_10 | 5′-AACUUGUCCUUAACGGUGCUC-3′ |
| 80 | APOC3_01 | 5′-UCACUGAGAAUACUGUCCCGUtcacctcatcccgcgaagc |
| 81 | APOC3_02 | 5′-UUCUUGUCCAGCUUUAUUGGGtcacctcatcccgcgaagc |
| 82 | APOC3_03 | 5′-UCCCUUUUAAGCAACCUACAGtcacctcatcccgcgaagc |
| 83 | APOC3_04 | 5′-UUGUCCUUAACGGUGCUCCAGtcacctcatcccgcgaagc |
| 84 | APOC3_05 | 5′-UCUUGUCCAGCUUUAUUGGGAtcacctcatcccgcgaagc |
| 85 | APOC3_06 | 5′-UUUUAAGCAACCUACAGGGGCtcacctcatcccgcgaagc |
| 86 | APOC3_07 | 5′-UGCUCCAGUAGUCUUUCAGGGtcacctcatcccgcgaagc |
| 87 | APOC3_08 | 5′-GAAUACUGUCCCUUUUAAGCAtcacctcatcccgcgaagc |
| 88 | APOC3_09 | 5′-UGGACUUGGGGUAUUGAGGUCtcacctcatcccgcgaagc |
| 89 | APOC3_10 | 5′-AACUUGUCCUUAACGGUGCUCtcacctcatcccgcgaagc |
| 90 | DGAT2_01 | 5′-CUCUGUAAAUUUGGAAGUGUCtcacctcatcccgcgaagc-3′ |
| 91 | DGAT2_02 | 5′-CACCAUGAGCUAGGUGGAGUAtcacctcatcccgcgaagc-3′ |
| 92 | DGAT2_03 | 5′-UUCCUGAAGUGACAAAGGAAAtcacctcatcccgcgaagc-3′ |
| 93 | DGAT2_04 | 5′-GACCACCAGGAACUAUAUCUUtcacctcatcccgcgaagc-3′ |
| 94 | DGAT2_05 | 5′-GUUCCAGAAAUACAUUGGUUUtcacctcatcccgcgaagc-3′ |
| 95 | DGAT2_06 | 5′-AACCGCAAGGGCUUUGUGAAAtcacctcatcccgcgaagc-3′ |
| 96 | DGAT2_07 | 5′-GAGCAAGAAGUUCCCAGGCAUtcacctcatcccgcgaagc-3′ |
| 97 | DGAT2_08 | 5′-CAGUAGUAGGCAUCUGGAAUGtcacctcatcccgcgaagc-3′ |
| 98 | DGAT2_09 | 5′-GUCAUGGGUGUCUGUGGGUUAtcacctcatcccgcgaagc-3 |
| 99 | DGAT2_10 | 5′-GCUCUGUAAAUUUGGAAGUGUtcacctcatcccgcgaagc-3′ |
| 100 | DGAT2_01 | 5′-CUCUGUAAAUUUGGAAGUGUC |
| 101 | DGAT2_02 | 5′-CACCAUGAGCUAGGUGGAGUA |
| 102 | DGAT2_03 | 5′-UUCCUGAAGUGACAAAGGAAA |
| 103 | DGAT2_04 | 5′-GACCACCAGGAACUAUAUCUU |
| 104 | DGAT2_05 | 5′-GUUCCAGAAAUACAUUGGUUU |
| 105 | DGAT2_06 | 5′-AACCGCAAGGGCUUUGUGAAA |
| 106 | DGAT2_07 | 5′-GAGCAAGAAGUUCCCAGGCAU |
| 107 | DGAT2_08 | 5′-CAGUAGUAGGCAUCUGGAAUG |
| 108 | DGAT2_09 | 5′-GUCAUGGGUGUCUGUGGGUUA |
| 109 | DGAT2_10 | 5′-GCUCUGUAAAUUUGGAAGUGU |
| 110 | DGAT2_01 | 5′-GACACUUCCAAAUUUACAGAG-3′ |
| 111 | DGAT2_02 | 5′-UACUCCACCUAGCUCAUGGUG-3′ |
| 112 | DGAT2_03 | 5′-UUUCCUUUGUCACUUCAGGAA-3′ |
| 113 | DGAT2_04 | 5′-AAGAUAUAGUUCCUGGUGGUC-3′ |
| 114 | DGAT2_05 | 5′-AAACCAAUGUAUUUCUGGAAC-3′ |
| 115 | DGAT2_06 | 5′-UUUCACAAAGCCCUUGCGGUU-3′ |
| 116 | DGAT2_07 | 5′-AUGCCUGGGAACUUCUUGCUC-3′ |
| 117 | DGAT2_08 | 5′-CAUUCCAGAUGCCUACUACUG-3′ |
| 118 | DGAT2_09 | 5′-UAACCCACAGACACCCAUGAC-3′ |
| 119 | DGAT2_10 | 5′-ACACUUCCAAAUUUACAGAGC-3′ |
| 120 | DGAT2_01 | 5′-GACACUUCCAAAUUUACAGAGtcacctcatcccgcgaagc |
| 122 | DGAT2_02 | 5′-UACUCCACCUAGCUCAUGGUGtcacctcatcccgcgaagc |
| 122 | DGAT2_03 | 5′-UUUCCUUUGUCACUUCAGGAAtcacctcatcccgcgaagc |
| 123 | DGAT2_04 | 5′-AAGAUAUAGUUCCUGGUGGUCtcacctcatcccgcgaagc |
| 124 | DGAT2_05 | 5′-AAACCAAUGUAUUUCUGGAACtcacctcatcccgcgaagc |
| 125 | DGAT2_06 | 5′-UUUCACAAAGCCCUUGCGGUUtcacctcatcccgcgaagc |
| 126 | DGAT2_07 | 5′-AUGCCUGGGAACUUCUUGCUCtcacctcatcccgcgaagc |
| 127 | DGAT2_08 | 5′-CAUUCCAGAUGCCUACUACUGtcacctcatcccgcgaagc |
| 128 | DGAT2_09 | 5′-UAACCCACAGACACCCAUGACtcacctcatcccgcgaagc |
| 129 | DGAT2_10 | 5′-ACACUUCCAAAUUUACAGAGCtcacctcatcccgcgaagc |

TABLE 3
Selection of DGAT2 siRNA sequences (SEQ ID NOs 131-170), PCSK9
(SEQ ID NO: 171-210 and ApoCIII (SEQ ID NO: 211 to 250)

| SEQ ID NO | sequence | |
|---|---|---|
| 131 | GACCACCAGGAACUAUAUCUU | sense sequence |
| 132 | GUUCCAGAAAUACAUUGGUUU | sense sequence |
| 133 | AACCGCAAGGGCUUUGUGAAA | sense sequence |
| 134 | GAGCAAGAAGUUCCCAGGCAU | sense sequence |
| 135 | CUUUGGAGAGAAUGAAGUGUA | sense sequence |
| 136 | CUUCGACAAGCACAAGACCAA | sense sequence |
| 137 | GCCGAUGGGUCCAGAAGAAGU | sense sequence |
| 138 | CUUCACUUGGCUGGUGUUUGA | sense sequence |
| 139 | CUCCUUUGGAGAGAAUGAAGU | sense sequence |
| 140 | UGCCAUCCUCAUGUACAUAUU | sense sequence |
| 141 | CCGCAAGGGCUUUGUGAAACU | sense sequence |
| 142 | AGCAAGAAGUUCCCAGGCAUA | sense sequence |
| 143 | AGUGUACAAGCAGGUGAUCUU | sense sequence |
| 144 | UGCUGACCACCAGGAACUAUA | sense sequence |
| 145 | CCGAUGGGUCCAGAAGAAGUU | sense sequence |
| 146 | UUUGGAGAGAAUGAAGUGUAC | sense sequence |
| 147 | UGGCGCUACUUUCGAGACUAC | sense sequence |
| 148 | AAUGCCUGUGUUGAGGGAGUA | sense sequence |
| 149 | AGUUCCAGAAAUACAUUGGUU | sense sequence |
| 150 | CAGAAGUGAGCAAGAAGUUCC | sense sequence |
| 151 | AAGAUAUAGUUCCUGGUGGUC | antisense sequence |
| 152 | AAACCAAUGUAUUUCUGGAAC | antisense sequence |
| 153 | UUUCACAAAGCCCUUGCGGUU | antisense sequence |
| 154 | AUGCCUGGGAACUUCUUGCUC | antisense sequence |
| 155 | UACACUUCAUUCUCUCCAAAG | antisense sequence |
| 156 | UUGGUCUUGUGCUUGUCGAAG | antisense sequence |
| 157 | ACUUCUUCUGGACCCAUCGGC | antisense sequence |
| 158 | UCAAACACCAGCCAAGUGAAG | antisense sequence |
| 159 | ACUUCAUUCUCUCCAAAGGAG | antisense sequence |
| 160 | AAUAUGUACAUGAGGAUGGCA | antisense sequence |
| 161 | AGUUUCACAAAGCCCUUGCGG | antisense sequence |
| 162 | UAUGCCUGGGAACUUCUUGCU | antisense sequence |
| 163 | AAGAUCACCUGCUUGUACACU | antisense sequence |
| 164 | UAUAGUUCCUGGUGGUCAGCA | antisense sequence |
| 165 | AACUUCUUCUGGACCCAUCGG | antisense sequence |
| 166 | GUACACUUCAUUCUCUCCAAA | antisense sequence |
| 167 | GUAGUCUCGAAAGUAGCGCCA | antisense sequence |
| 168 | UACUCCCUCAACACAGGCAUU | antisense sequence |
| 169 | AACCAAUGUAUUUCUGGAACU | antisense sequence |
| 170 | GGAACUUCUUGCUCACUUCUG | antisense sequence |
| 171 | CCUCAUAGGCCUGGAGUUUAU | sense sequence |
| 172 | AGGCCUGGAGUUUAUUCGGAA | sense sequence |
| 173 | CCCUCAUAGGCCUGGAGUUUA | sense sequence |
| 174 | AGGUCUGGAAUGCAAAGUCAA | sense sequence |
| 175 | GGCCUGGAGUUUAUUCGGAAA | sense sequence |
| 176 | CAGGUCUGGAAUGCAAAGUCA | sense sequence |
| 177 | CCUCACCAAGAUCCUGCAUGU | sense sequence |
| 178 | ACCCUCAUAGGCCUGGAGUUU | sense sequence |
| 179 | CACCAGCAUACAGAGUGACCA | sense sequence |
| 180 | AUCUCCUAGACACCAGCAUAC | sense sequence |
| 181 | UCCUAGACACCAGCAUACAGA | sense sequence |
| 182 | CUGGAGUUUAUUCGGAAAAGC | sense sequence |
| 183 | GCCUGGAGUUUAUUCGGAAAA | sense sequence |
| 184 | GAGGCAGAGACUGAUCCACUU | sense sequence |
| 185 | UAGGCCUGGAGUUUAUUCGGA | sense sequence |
| 186 | CACUUCUCUGCCAAAGAUGUC | sense sequence |
| 187 | AUGCAAAGUCAAGGAGCAUGG | sense sequence |
| 188 | GGUCAUGGUCACCGACUUCGA | sense sequence |
| 189 | GGCAGCUGUUUUGCAGGACUG | sense sequence |
| 190 | GGGCAGGUUGGCAGCUGUUUU | sense sequence |
| 191 | AUAAACUCCAGGCCUAUGAGG | antisense sequence |
| 192 | UUCCGAAUAAACUCCAGGCCU | antisense sequence |
| 193 | UAAACUCCAGGCCUAUGAGGG | antisense sequence |
| 194 | UUGACUUUGCAUUCCAGACCU | antisense sequence |
| 195 | UUUCCGAAUAAACUCCAGGCC | antisense sequence |
| 196 | UGACUUUGCAUUCCAGACCUG | antisense sequence |
| 197 | ACAUGCAGGAUCUUGGUGAGG | antisense sequence |
| 198 | AAACUCCAGGCCUAUGAGGGU | antisense sequence |
| 199 | UGGUCACUCUGUAUGCUGGUG | antisense sequence |
| 200 | GUAUGCUGGUGUCUAGGAGAU | antisense sequence |
| 201 | UCUGUAUGCUGGUGUCUAGGA | antisense sequence |
| 202 | GCUUUUCCGAAUAAACUCCAG | antisense sequence |
| 203 | UUUUCCGAAUAAACUCCAGGC | antisense sequence |
| 204 | AAGUGGAUCAGUCUCUGCCUC | antisense sequence |
| 205 | UCCGAAUAAACUCCAGGCCUA | antisense sequence |
| 206 | GACAUCUUUGGCAGAGAAGUG | antisense sequence |
| 207 | CCAUGCUCCUUGACUUUGCAU | antisense sequence |
| 208 | UCGAAGUCGGUGACCAUGACC | antisense sequence |
| 209 | CAGUCCUGCAAAACAGCUGCC | antisense sequence |
| 210 | AAAACAGCUGCCAACCUGCCC | antisense sequence |
| 211 | CUGGAGCACCGUUAAGGACAA | sense sequence |
| 212 | CCCUGAAAGACUACUGGAGCA | sense sequence |
| 213 | GAGCACCGUUAAGGACAAGUU | sense sequence |
| 214 | ACUGGAGCACCGUUAAGGACA | sense sequence |
| 215 | CCUGAAAGACUACUGGAGCAC | sense sequence |
| 216 | AAGACUACUGGAGCACCGUUA | sense sequence |
| 217 | CAGUUCCCUGAAAGACUACUG | sense sequence |
| 218 | GGUGACCGAUGGCUUCAGUUC | sense sequence |
| 219 | GGGUGACCGAUGGCUUCAGUU | sense sequence |
| 220 | ACUACUGGAGCACCGUUAAGG | sense sequence |
| 221 | GACUACUGGAGCACCGUUAAG | sense sequence |
| 222 | UUCAGUUCCCUGAAAGACUAC | sense sequence |
| 223 | GUUCCCUGAAAGACUACUGGA | sense sequence |
| 224 | UGGAGCACCGUUAAGGACAAG | sense sequence |
| 225 | CGCCACCAAGACCGCCAAGGA | sense sequence |
| 226 | GGGCUGGGUGACCGAUGGCUU | sense sequence |
| 227 | GCCACCAAGACCGCCAAGGAU | sense sequence |
| 228 | AGACUACUGGAGCACCGUUAA | sense sequence |
| 229 | CCACCAAGACCGCCAAGGAUG | sense sequence |
| 230 | UCCCUGAAAGACUACUGGAGC | sense sequence |
| 231 | UUGUCCUUAACGGUGCUCCAG | antisense sequence |
| 232 | UGCUCCAGUAGUCUUUCAGGG | antisense sequence |
| 233 | AACUUGUCCUUAACGGUGCUC | antisense sequence |
| 234 | UGUCCUUAACGGUGCUCCAGU | antisense sequence |
| 235 | GUGCUCCAGUAGUCUUUCAGG | antisense sequence |
| 236 | UAACGGUGCUCCAGUAGUCUU | antisense sequence |
| 237 | CAGUAGUCUUUCAGGGAACUG | antisense sequence |
| 238 | GAACUGAAGCCAUCGGUCACC | antisense sequence |
| 239 | AACUGAAGCCAUCGGUCACCC | antisense sequence |
| 240 | CCUUAACGGUGCUCCAGUAGU | antisense sequence |
| 241 | CUUAACGGUGCUCCAGUAGUC | antisense sequence |
| 242 | GUAGUCUUUCAGGGAACUGAA | antisense sequence |
| 243 | UCCAGUAGUCUUUCAGGGAAC | antisense sequence |
| 244 | CUUGUCCUUAACGGUGCUCCA | antisense sequence |
| 245 | UCCUUGGCGGUCUUGGUGGCG | antisense sequence |
| 246 | AAGCCAUCGGUCACCCAGCCC | antisense sequence |
| 247 | AUCCUUGGCGGUCUUGGUGGC | antisense sequence |
| 248 | UUAACGGUGCUCCAGUAGUCU | antisense sequence |
| 249 | CAUCCUUGGCGGUCUUGGUGG | antisense sequence |
| 250 | GCUCCAGUAGUCUUUCAGGGA | antisense sequence |

TABLE 4
siRNAs pairs used in silencing of APOC3 and DGAT 2 gene expression in
HEP2G cells in vitro

| Name | | SEQ | |
|---|---|---|---|
| (SEQ ID | | ID | |
| NO) | Sense | NO | Antisense |
| APOC3_01 | 5′- | 70 | 5′- |
| (50) | ACGGGACAGUAUUCUCAGUNAtcacctcatcccgcgaagc- | | UCACUGAGAAUACUGUCCCGU- |
| | 3′ | | 3′ |
| APOC3_02 | 5′- | 71 | 5′- |
| (51) | CCCAAUAAAGCUGGACAAGAAtcacctcatcccgcgaagc-3′ | | UUCUUGUCCAGCUUUAUUGGG- |
| | | | 3′ |
| APOC3_03 | 5′- | 72 | 5′- |
| (52) | CUGUAGGUUGCUUAAAAGGGAtcacctcatcccgcgaagc- | | UCCCUUUUAAGCAACCUACAG- |
| | 3′ | | 3′ |
| APOC3_04 | 5′- | 73 | 5′- |
| (53) | CUGGAGCACCGUUAAGGACAAtcacctcatcccgcgaagc- | | UUGUCCUUAACGGUGCUCCAG- |
| | 3′ | | 3′ |
| APOC3_05 | 5′- | 74 | 5′- |
| (54) | UCCCAAUAAAGCUGGACAAGAtcacctcatcccgcgaagc-3′ | | UCUUGUCCAGCUUUAUUGGGA- |
| | | | 3′ |
| APOC3_06 | 5′- | 75 | 5′- |
| (55) | GCCCCUGUAGGUUGCUUAAAAtcacctcatcccgcgaagc- | | UUUUAAGCAACCUACAGGGGC- |
| | 3′ | | 3′ |
| APOC3_07 | 5′- | 76 | 5′- |
| (56) | CCCUGAAAGACUACUGGAGCAtcacctcatcccgcgaagc- | | UGCUCCAGUAGUCUUUCAGGG- |
| | 3′ | | 3′ |
| APOC3_08 | 5′- | 77 | 5′- |
| (57) | UGCUUAAAAGGGACAGUAUUCtcacctcatcccgcgaagc- | | GAAUACUGUCCCUUUUAAGCA- |
| | 3′ | | 3′ |
| APOC3_09 | 5′- | 78 | 5′- |
| (58) | GACCUCAAUACCCCAAGUCCAtcacctcatcccgcgaagc-3′ | | UGGACUUGGGGUAUUGAGGUC |
| | | | -3′ |
| APOC3_10 | 5′- | 79 | 5′- |
| (59) | GAGCACCGUUAAGGACAAGUUtcacctcatcccgcgaagc- | | AACUUGUCCUUAACGGUGCUC- |
| | 3′ | | 3′ |
| DGAT2_01 | 5′- | 110 | 5′- |
| (90) | CUCUGUAAAUUUGGAAGUGUCtcacctcatcccgcgaagc- | | GACACUUCCAAAUUUACAGAG- |
| | 3′ | | 3′ |
| DGAT2_02 | 5′- | 111 | 5′- |
| (91) | CACCAUGAGCUAGGUGGAGUAtcacctcatcccgcgaagc- | | UACUCCACCUAGCUCAUGGUG- |
| | 3′ | | 3′ |
| DGAT2_03 | 5′- | 112 | 5′- |
| (92) | UUCCUGAAGUGACAAAGGAAAtcacctcatcccgcgaagc- | | UUUCCUUUGUCACUUCAGGAA- |
| | 3′ | | 3′ |
| DGAT2_04 | 5′- | 113 | 5′- |
| (93) | GACCACCAGGAACUAUAUCUUtcacctcatcccgcgaagc- | | AAGAUAUAGUUCCUGGUGGUC- |
| | 3′ | | 3′ |
| DGAT2_05 | 5′- | 114 | 5′- |
| (103) | GUUCCAGAAAUACAUUGGUUUtcacctcatcccgcgaagc- | | AAACCAAUGUAUUUCUGGAAC- |
| | 3′ | | 3′ |
| DGAT2_06 | 5′- | 115 | 5′- |
| (105) | AACCGCAAGGGCUUUGUGAAAtcacctcatcccgcgaagc- | | UUUCACAAAGCCCUUGCGGUU- |
| | 3′ | | 3′ |
| DGAT2_07 | 5′- | 116 | 5′- |
| (106) | GAGCAAGAAGUUCCCAGGCAUtcacctcatcccgcgaagc- | | AUGCCUGGGAACUUCUUGCUC- |
| | 3′ | | 3′ |
| DGAT2_08 | 5′- | 117 | 5′- |
| (107) | CAGUAGUAGGCAUCUGGAAUGtcacctcatcccgcgaagc- | | CAUUCCAGAUGCCUACUACUG- |
| | 3′ | | 3′ |
| DGAT2_09 | 5′- | 118 | 5′- |
| (108) | GUCAUGGGUGUCUGUGGGUUAtcacctcatcccgcgaagc- | | UAACCCACAGACACCCAUGAC-3′ |
| | 3′ | | |
| DGAT2_10 | 5′- | 119 | 5′- |
| (109) | GCUCUGUAAAUUUGGAAGUGUtcacctcatcccgcgaagc- | | ACACUUCCAAAUUUACAGAGC- |
| | 3′ | | 3′ |

### Animals

Sufficient C57BL/6 mice or transgenic equivalent were obtained from an approved source to provide 20 healthy male animals (ApoB pilot study). Animals are in the target weight range of 20 to 30 g at dosing. Mice are uniquely numbered by tail marking. Numbers are allocated randomly. Cages are coded by cards giving information including study number and animal number. The study room is identified by a card giving information including room number and study number. On receipt, all animals were examined for external signs of ill health. Unhealthy animals where be excluded from the study. The animals were acclimatised for a minimum period of 5 days. Where practicable, without jeopardising the scientific integrity of the study, animals were handled as much as possible. A welfare inspection was performed before the start of dosing to ensure their suitability for the study.

The mice were kept in rooms thermostatically maintained at a temperature of 20 to 24° C., with a relative humidity of between 45 and 65%, and exposed to fluorescent light (nominal 12 hours) each day. Temperature and relative humidity are recorded on a daily basis. The facility is designed to give a minimum of 15 air-changes/hour. Except when in metabolism cages or recovering from surgery, mice were housed up to 5 per cage according to sex, in suitable solid floor cages, containing suitable bedding.

Cages conform to the ‘Code of Practice for the Housing and Care of Animals Bred, Supplied or Used for Scientific Purposes' (Home Office, London, 2014). In order to enrich both the environment and the welfare of the animals, they were provided with wooden Aspen chew blocks and polycarbonate tunnels. The supplier provided certificates of analysis for each batch of blocks used. All animals will be allowed free access to 5LF2 EU Rodent Diet 14%. The diet supplier provided an analysis of the concentration of certain contaminants and some nutrients for each batch used. All animals were allowed free access to mains water from bottles attached to the cages. Periodic analysis of the mains supply is undertaken.

All procedures to be carried out on live animals as part of this study will be subject to provisions of United Kingdom National Law, the Animals (Scientific Procedures) Act 1986.

All animals were examined at the beginning and the end of the working day, to ensure that they are in good health. Any animal, which shows marked signs of ill health, were isolated. Moribund animals or those in danger of exceeding the severity limits imposed by the relevant Home Office Licence were killed.

### Preparation of Formulations

Test substances were diluted in 0.9% saline to provided concentrations of 25 mg/mL and 0.6 mg/mL for the intravenous and subcutaneous doses of lipoprotein (a) or ApoB Crook-siRNA GalNAc-unconjugated and conjugate respectively. The formulations were gently vortexed as appropriate until the test substances are fully dissolved.

For PCSK9, lyophilised siRNA compounds were dissolved and subsequently diluted in nuclease-free PBS (neutral pH).

The resulting formulation(s) were assessed by visual inspection only and categorised accordingly:
(1) Clear solution
(2) Cloudy suspension, no particles visible
(3) Visible particles

After use, formulations were stored refrigerated nominally at 2-8° C. For long-term storage, formulations were stored at −20 C or −80 C

### Dosing Details

Each animal received either a single intravenous dose of the lipoprotein (a) or ApoB Crook-siRNA GalNAc-unconjugated or a single subcutaneous dose lipoprotein (a) or ApoB Crook-siRNA GalNAc-conjugate. The intravenous dose was administered as a bolus into the lateral tail vein at a volume of 2 mL/kg. The subcutaneous dose was administered into the subcutaneous space at a volume of 5 mL/kg.

For PCSK9 test substances, each animal received a single subcutaneous injection at a dosing volume of 5 ml/kg.

### Body Weights

As a minimum, body weights were recorded the day after arrival and before dose administration. Additional determinations were made, if required.

### Sample Storage

Samples were uniquely labelled with information including, where appropriate: study number; sample type; dose group; animal number/Debra code; (nominal) sampling time; storage conditions. Samples were stored at <−50° C.

### Pharmacokinetic Investigation

### Designation of Dose Groups

Animals were assigned to dose groups as follows:

| | | | Dose | Number of |
|---|---|---|---|---|
| Dose | | | level | animals |
| Group | Dose route | Test Substance | mg/kg | Male |
| A | Subcutaneous | Lipoprotein (a) or ApoB | 5 | 5 |
| | | Crook-siRNA GalNAc- | | |
| | | conjugate | | |
| B | Subcutaneous | Saline control | 0 | 5 |
| C | Intravenous | Lipoprotein (a) or ApoB | 50 | 5 |
| | (bolus) | Crook-siRNA | | |
| | | unconjugated | | |
| D | Intravenous | Saline control | 0 | 5 |
| | (bolus) | | | |

### PCSK9

Test substances were dissolved in nuclease-free PBS (neutral pH) to obtain concentrations of 0.4 mg/mL or 2 mg/mL to provide doses of 2 mg/kg and 10 mg/kg, respectively, when given subcutaneously in a 5 mL/kg dosing volume.

For PCSK9, each animal received a single subcutaneous dose of either the GalNAc-conjugated PCSK9 Crook siRNA, or GalNAc-conjugated PCSK9 without Crook, and sacrificed at either Day2 (48 hrs) or Day 7 (168 hrs) to determine liver PCSK9 mRNA silencing. Samples are obtained either via tail bleed or cardiac puncture at conclusion.

For Each of the PCSK9 Crook siRNA

| | | |
|---|---|---|
| 10 mice | SC | GalNAc-conjugated PCSK9 crook-siRNA at 2 mg/kg |
| 10 mice | SC | GalNAc-conjugated PCSK9 crook-siRNA at 10 mg/kg |
| 10 mice | SC | GalNAc-conjugated PCSK9 ‘No crook’-siRNA at 2 mg/kg |
| 10 mice | SC | GalNAc-conjugated PCSK9 ‘No crook’-siRNA at 10 mg/kg |
| 10 mice | SC | PBS control |

### Blood Sampling

Serial blood samples of (nominally 100 μL, dependent on bodyweight) were collected by tail nick at the following times: 0, 48 and 96* hours post dose. Animals were terminally anaesthetised using isoflurane and a final sample (nominally 0.5 mL) was collected by cardiac puncture.

Blood samples were collected in to a K2EDTA microcapillary tube (tail nick) or a K2EDTA blood tube (cardiac puncture) and placed on ice until processed. Blood was centrifuged (1500 g, 10 min, 4° C.) to produce plasma for analysis. The bulk plasma was divided into two aliquots of equal volume. The residual blood cells were discarded. The acceptable time ranges for blood sample collections are summarised in the following table. Actual sampling times were recorded for all matrices.

| Scheduled Collection | Acceptable Time | |
|---|---|---|
| Time | Range | |
| 0-15 minutes | ±1 | minute |
| 16-30 minutes | ±2 | minutes |
| 31-45 minutes | ±3 | minutes |
| 46-60 minutes | ±4 | minutes |
| 61 minutes-2 hours | ±5 | minutes |
| 2 hours 1 minute-8 hours | ±10 | minutes |
| 8 hours 1 minute-12 hours | ±15 | minutes |
| 12 hours onwards | ±30 | minutes |

Where a scheduled collection time is outside the acceptable range, the actual blood collection time was reported for inclusion in any subsequent PK analysis

For serum collection, blood (>300 ul) is placed into serum tubes at ambient temperature and allowed to clot, then centrifuged at 10,000 rpm for 5 mins.

### Animal Fate

Animals were anaesthetised via an intraperitoneal injection of Sodium Pentobarbitone prior to terminal blood sampling and sacrificed by isoflurane administration.

### Tissue Collection

The liver was removed from all animals (Groups A-D) and placed into a pre-weighed tube. The tissue samples were homogenised with 5 parts RNAlater to 1 part tissue using the UltraTurrax homogenisation probe. The following tissues were excised from animals in lipoprotein (a) or ApoB treated groups (Groups A & C) and placed into a pre-weighed pot:
Spleen
Brain
Heart
Lung Lobes
Skin (Inguinal region ca. 25 mm²)

Tissues were snap frozen in liquid nitrogen to avoid RNase activity. Tissues are stored at <−50° C. (nominally −80° C.).

### For PCSK9 In Vivo Study: Liver Processing for RT-qPCR

Animals were sacrificed and livers harvested and snap frozen in liquid nitrogen. Whole liver was ground and tissue lysates were prepared for assessment of PCSK9 mRNA expression by RT-qPCR as described below.
Total RNA was extracted from 10 mg of ground liver tissue using the GenElute™ Total RNA Purification Kit (RNB100-100RXN).
Duplex RT-qPCR was performed using the ThermoFisher TaqMan Fast 1-Step Master Mix with TaqMan probes for mouse GAPDH (VIC_PL) and PCSK9 (FAM).
Relative quantification (RQ) of PCSK9 mRNA was determined using the ΔΔCT method, where GAPDH was used as internal control and the expression changes of the target gene were normalized to the vehicle control (PBS).

### Immunoassays

Plasma lipoprotein (a) or ApoB levels were measured via enzyme-linked immunosorbent assay (ELISA) using the commercial mouse lipoprotein (a) or ApoB detection kit from Elabscience Biotechnology Inc. Plasma samples were stored at −80° C. prior to analysis, thawed on ice and centrifuged at 13,000 rpm for 5 minutes prior to aliquots being diluted in Assay Buffer and applied to the ELISA plate. The lipoprotein (a) or ApoB assay kit uses a sandwich ELISA yielding a colorimetric readout, measured at OD450. Samples from each animal at specific time points (0 hours and 96 hours) were assayed in duplicate and measurements were recorded as micrograms lipoprotein (a) or ApoB per ml of plasma based on the standard curve reagents supplied with the kit. All data points were measured with a coefficient of variation <20%. Change in lipoprotein (a) or ApoB level for each animal was calculated by subtracting the 0 hour value from the 96 hour value and expressed as a percentage. The range of % change values were collated for each study group and statistical analysis applied using the two-tailed paired t test algorithm.

### Example 1

A pilot in vivo mouse experiment was performed to assess activity of GalNAc-conjugated Crook anti-mouse ApoB siRNA compared to control siRNA constructs. Conjugated (GalNAc) and unconjugated (without GalNAc) versions of ApoB Crook siRNA were administered to adult male wild-type (WT) C57BL/6 mice by sub-cutaneous (SC) and intravenous (IV) routes, respectively described previously in Material & Methods section.

Blood plasma ApoB was measured by ELISA (described earlier) at time 0 (prior to administration of siRNA construct) and at 96 hours following siRNA construct administration, as indicated in the four Treatment groups (5 mice per group) as detailed above under Dosing Details.

Plasma ApoB levels (micrograms/ml) from 5 mice in each treatment group, were used to calculate a mean ApoB value+/−standard error of the mean (SEM). Change in plasma ApoB level after 96 hours following SC administration of GalNAc-conjugated Crook siRNA was compared to levels in mice receiving either control (i) vehicle saline, or (ii) unconjugated siRNA with Crook. Statistical analysis was applied using the two-tailed paired T test algorithm.

With reference to FIG. 1 (a), plasma ApoB levels (micrograms/ml) of mice 96 hours following treatment with GalNAc-conjugated ApoB Crook siRNA were compared with the control treatment group administered with saline. Statistical analysis was applied using the two-tailed paired T test algorithm. Results show a substantive reduction in mean plasma ApoB levels in mice treated with GalNAc-conjugated Crook siRNA, compared to control. However, it just fails significance (p=0.11), most likely due to small sample size and variation in ApoB levels between control animals.

With reference to FIG. 1 (b), plasma ApoB levels (micrograms/ml) measured 96 hours following administration of GalNAc-conjugated ApoB Crook siRNA were compared to the control group, treated with siRNA construct unconjugated (without GalNAc) ApoB Crook siRNA. Statistical analysis was applied using the two-tailed paired T test algorithm.

Results show a highly significant reduction in plasma ApoB levels in this GalNAc-conjugated Crook siRNA treatment group when compared to control unconjugated siRNA with Crook (P=0.00435832).

### Example 2

Our optimization assays demonstrated that LP(a) mRNA cannot be detected in HepG2 cells by RT-qPCR using specific TaqMan probes. The most likely explanation is that LP(a) is not expressed in HepG2, which is consistent with publicly available expression data (online at proteinatlas.org/ENSG00000198670-LP(A)/cell). The RT4 cell line has been reported to express high levels of LP(a), and therefore this cell line was evaluated for its suitability for the study of LP(a) expression levels.

RT4 cells were transfected with toxic siTOX and ON TARGETplus siRNAs Non-Targeting or targeting PLK1, LP(a) or PCSK9. 72 h post-transfection, cells were processed to evaluate cell viability (FIG. 2) or LP(a) and PCSK9 expression (FIG. 3). The robust cell death induced by siTOX and siRNA targeting the essential gene PLK1 (FIG. 2) indicate the high transfection efficiency. This is further confirmed by the decreased PCSK9 and LP(a) expression following treatment with the corresponding OT+ siRNAs (FIG. 3). LP(a) mRNA could readily be detected by TaqMan probes, confirming the gene is expressed in RT4. These results suggest that RT4 is a suitable model to evaluate the LP(a) crook siRNAs library.

In each assay plate, HepG2 were transfected with OT+ siRNA Non-Targeting, targeting the essential gene PLK1 or targeting LP(a). The high level of cell death following treatment with the PLK1 siRNA indicates the transfection efficiency (FIG. 4). Although significant, the decrease in LP(a) expression following treatment with LP(a) OT+ siRNA was only modest (FIG. 5).

The toxicity induced by the crook siRNAs targeting LP(a) and NEG controls in RT4 was substantially higher than observed in HepG2. This can be illustrated by the GAPDH CT values (FIG. 6). This toxicity greatly impairs the data quality and therefore the ability of the data to be used to calculate the changes in LP(a) expression following siRNA treatment. In order to counter this and enable the data to be presented clearly, it was decided that when GAPDH CT value increases by more than 3.5 between treatment at 0.39 nM and treatment at 25 nM (corresponding to a decreased number of cells of more than 90%), the mRNA levels for the corresponding siRNA would not be analysed. This process had led to the exclusion of data from the two NEG controls and 6 of the 16 Crook siRNAs. By comparison, no Crook siRNAs targeting PCSK9 led to an increase in GAPDH CT of more than 3 in HepG2 cells.

The knock-down activity measured for the 10 LP(a) crook siRNA displaying no or limited toxicity is shown in FIG. 7. Overall, the knockdown efficiency of the siRNAs targeting LP(a) were not as effective at the same dose as those targeting PCSK9, which were tested in the HepG2 cell line.

LP7, LP11 and LP12 showed a knock-down efficiency above 50% at 25 nM. This level of knockdown appears to be substantially better than that observed for the OnTARGET Plus siRNA targeting LP(a).

### Example 3

### In Vivo Silencing of PCSK9

With reference to FIG. 8, an in vivo mouse study was performed to assess knockdown activity of GalNAc-conjugated Crook anti-mouse PCSK9 siRNA (Compound H) compared to its ‘no Crook’ siRNA control (Compound A); see FIG. 9. Test siRNA compounds were administered to adult male wild-type (WT) C57BL/6 mice by sub-cutaneous (SC) injection at either 2 or 10 mg/kg (5 replicates per treatment group). Vehicle (PBS) group of 5 replicates served as a negative control.

After 48 hours, mice were sacrificed and whole livers harvested for quantification of PCSK9 mRNA by RT-qPCR as described in earlier in Material & Methods section.

Compound H (at 2 mg/kg) shows approx. 50% knockdown of liver PCSK9 mRNA, 48 hrs after SC injection, compared to Compound A and Vehicle control. Statistical analysis was applied using the two-tailed paired T test algorithm.

Results show a highly significant reduction in liver PCSK9 mRNA plasma in GalNAc-conjugated PCSK9 Crook siRNA treatment group (H) when compared to GalNAc-conjugated PCSK9 ‘no Crook’ siRNA treatment group (A) and Vehicle (PBS) control (p<0.001 vs Vehicle)

### Example 4

With reference to Table 5, an RNAi screen in HepG2 cells was performed to evaluate a custom library of 10 “Crook” siRNAs targeting ApoC3 (listed in Table 4). HepG2 cells were reverse transfected with the 10 siRNAs. 72 hr post transfection, ApoC3 mRNA levels were quantified by duplex RT-qPCR, normalizing the ApoC3 mRNA levels to the levels of the housekeeping reference gene GAPDH mRNA. Overall, all the siRNA sequences (ApoC3-01 to ApoC3-10) displayed over 80% knockdown at 25 and 6.25 nM siRNA concentration compared to no treatment. Five sequences (ApoC3-01, ApoC3-05, ApoC3-07, APOC3-09, APOC3-10) showed more than 95% KD at 25 nM when compared to no treatment control. Table 5 In vitro Knockdown % of APOC3 mRNA compared to no treated control in HepG2 cells following 72 hours transfection with 4-point 4-fold dilution series of crooked siRNAs.

| Sequence ID | Concentration (nM) | KD of APOC3 (%) |
|---|---|---|
| APOC3_01 | 25 | 97 |
| APOC3_01 | 6.25 | 88 |
| APOC3_01 | 1.56 | 88 |
| APOC3_01 | 0.39 | 82 |
| APOC3_02 | 25 | 93 |
| APOC3_02 | 6.25 | 84 |
| APOC3_02 | 1.56 | 82 |
| APOC3_02 | 0.39 | 74 |
| APOC3_03 | 25 | 93 |
| APOC3_03 | 6.25 | 84 |
| APOC3_03 | 1.56 | 70 |
| APOC3_03 | 0.39 | 60 |
| APOC3_04 | 25 | 92 |
| APOC3_04 | 6.25 | 83 |
| APOC3_04 | 1.56 | 73 |
| APOC3_04 | 0.39 | 64 |
| APOC3_05 | 25 | 97 |
| APOC3_05 | 6.25 | 84 |
| APOC3_05 | 1.56 | 75 |
| APOC3_05 | 0.39 | 73 |
| APOC3_06 | 25 | 92 |
| APOC3_06 | 6.25 | 91 |
| APOC3_06 | 1.56 | 72 |
| APOC3_06 | 0.39 | 67 |
| APOC3_07 | 25 | 99 |
| APOC3_07 | 6.25 | 93 |
| APOC3_07 | 1.56 | 77 |
| APOC3_07 | 0.39 | 70 |
| APOC3_08 | 25 | 76 |
| APOC3_08 | 6.25 | 64 |
| APOC3_08 | 1.56 | 49 |
| APOC3_08 | 0.39 | 44 |
| APOC3_09 | 25 | 98 |
| APOC3_09 | 6.25 | 89 |
| APOC3_09 | 1.56 | 81 |
| APOC3_09 | 0.39 | 71 |
| APOC3_10 | 25 | 99 |
| APOC3_10 | 6.25 | 94 |
| APOC3_10 | 1.56 | 84 |
| APOC3_10 | 0.39 | 76 |

### Example 5

With reference to Table 6, an RNAi screen in HepG2 cells was performed to evaluate a custom library of 10 “Crook” siRNAs targeting DGAT2 (listed in Table 4). HepG2 cells were reverse transfected with the 10 siRNAs. 72 hr post transfection, DGAT2 mRNA levels were quantified by duplex RT-qPCR, normalizing the DGAT2 mRNA levels to the levels of the housekeeping reference gene GAPDH mRNA. Four siRNA sequences (DGAT2-01, DGAT2-04, DGAT2-09, DGAT2-10) showed more than 80% knockdown of DGAT2 mRNA at the highest dose (25 nM).

TABLE 6
Knockdown % of DGAT2 mRNA compared to no treated control
in HepG2 cells following 72 hours transfection with
4-point 4-fold dilution series of crooked siRNAs

| Sequence ID | Concentration (nM) | KD of DGAT2 (%) |
|---|---|---|
| DGAT2_01 | 25 | 84 |
| DGAT2_01 | 6.25 | 77 |
| DGAT2_01 | 1.56 | 69 |
| DGAT2_01 | 0.39 | 67 |
| DGAT2_02 | 25 | 46 |
| DGAT2_02 | 6.25 | 44 |
| DGAT2_02 | 1.56 | 43 |
| DGAT2_02 | 0.39 | 39 |
| DGAT2_03 | 25 | 23 |
| DGAT2_03 | 6.25 | 26 |
| DGAT2_03 | 1.56 | 35 |
| DGAT2_03 | 0.39 | 24 |
| DGAT2_04 | 25 | 88 |
| DGAT2_04 | 6.25 | 78 |
| DGAT2_04 | 1.56 | 51 |
| DGAT2_04 | 0.39 | 41 |
| DGAT2_05 | 25 | 43 |
| DGAT2_05 | 6.25 | 12 |
| DGAT2_05 | 1.56 | 40 |
| DGAT2_05 | 0.39 | 32 |
| DGAT2_06 | 25 | 37 |
| DGAT2_06 | 6.25 | 35 |
| DGAT2_06 | 1.56 | 32 |
| DGAT2_06 | 0.39 | 28 |
| DGAT2_07 | 25 | 55 |
| DGAT2_07 | 6.25 | 49 |
| DGAT2_07 | 1.56 | 35 |
| DGAT2_07 | 0.39 | 31 |
| DGAT2_08 | 25 | 55 |
| DGAT2_08 | 6.25 | 39 |
| DGAT2_08 | 1.56 | 32 |
| DGAT2_08 | 0.39 | 31 |
| DGAT2_09 | 25 | 89 |
| DGAT2_09 | 6.25 | 76 |
| DGAT2_09 | 1.56 | 67 |
| DGAT2_09 | 0.39 | 58 |
| DGAT2_10 | 25 | 81 |
| DGAT2_10 | 6.25 | 48 |
| DGAT2_10 | 1.56 | 47 |
| DGAT2_10 | 0.39 | 50 |

### REFERENCES

Nair, J. K., Willoughby, J. L., Chan, A., Charisse, K., Alarn, M. R., Wang, Q., Hoekstra, M., Kandasamy, P., Kel'in, A. V., Milstein, S. and Taneja, N., 2014. Multivalent N-acetylgalactosamine-conjugated siRNA localizes in hepatocytes and elicits robust RNAi-mediated gene silencing. Journal of the American Chemical Society, 136(49), pp. 16958-16961.
Soutschek, J., Akinc, A., Bramlage, B., Charisse, K., Constien, R., Donoghue, M., Elbashir, S., Geick, A., Hadwiger, P., Harborth, J. and John, M., 2004. Therapeutic silencing of an endogenous gene by systemic administration of modified siRNAs. Nature, 432(7014), p. 173

1. A nucleic acid molecule comprising:
a first part that comprises a double stranded inhibitory ribonucleic acid (RNA) molecule comprising a sense strand and an antisense strand; and
a second part that comprises a single stranded deoxyribonucleic acid (DNA) molecule, wherein 5′ end of said single stranded DNA molecule is covalently linked to the 3′ end of the sense strand of the double stranded inhibitory RNA molecule or wherein 5′ end of the single stranded DNA molecule is covalently linked to 3′ of the antisense strand of the double stranded inhibitory RNA molecule, characterized in that the double stranded inhibitory RNA comprises a sense nucleotide sequence that encodes a part of a cardiovascular gene target associated with cardiovascular disease wherein said gene target is not apolipoprotein B and proprotein convertase subtilisin kexin type 9, or a polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

2. The nucleic acid molecule according to claim 1 wherein said loop domain comprises the nucleotide sequence GCGAAGC.

3. The nucleic acid molecule according to claim 1, wherein said single stranded DNA molecule comprises the nucleotide sequence
(SEQ ID NO: 251)
TCACCTCATCCCGCGAAGC.

4. The nucleic acid molecule according to claim 1, wherein said inhibitory RNA molecule comprises a two-nucleotide overhang.

5. The nucleic acid molecule according to claim 1, wherein said sense strand and/or said antisense strand comprises internucleotide phosphorothioate linkages.

6. The nucleic acid molecule according to claim 1, wherein:
said single stranded DNA molecule comprises one or more internucleotide phosphorothioate linkages;
said nucleic acid molecule comprises a vinylphosphonate modification; and/or
said double stranded inhibitory RNA molecule is between 19 and 23 nucleotides in length.

7-9. (canceled)

10. The nucleic acid molecule according to claim 1, wherein said cardiovascular gene target is Human Lipoprotein (a).

11. The nucleic acid molecule according to claim 10 wherein said double stranded inhibitory RNA molecule comprises an antisense nucleotide sequence selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 and 34.

12. The nucleic acid molecule according to claim 10 wherein:
said double stranded inhibitory RNA molecule comprises an antisense nucleotide sequence comprising SEQ ID NO: 41 and a sense nucleotide sequence comprising SEQ ID NO: 49, wherein said single stranded DNA molecule is covalently linked to the 3′ end of the sense strand of the double stranded inhibitory RNA molecule;
said double stranded inhibitory RNA molecule comprises an antisense nucleotide sequence comprising SEQ ID NO: 1 and a sense nucleotide sequence comprising SEQ ID NO: 44, wherein said single stranded DNA molecule is covalently linked to the 3′ end of the antisense strand of the double stranded inhibitory RNA molecule; or
said double stranded inhibitory RNA molecule comprises an antisense nucleotide sequence comprising SEQ ID NO: 5 and a sense nucleotide sequence comprising SEQ ID NO: 46, wherein said single stranded DNA molecule is covalently linked to the 3′ end of the antisense strand of the double stranded inhibitory RNA molecule.

13-14. (canceled)

15. The nucleic acid molecule according to claim 1, wherein said cardiovascular gene target is Human Apolipoprotein C III (Apo C III).

16. The nucleic acid molecule according to claim 15 wherein:
said nucleic acid molecule comprises an RNA strand comprising a nucleotide sequence selected from the group consisting of: SEQ ID NO: 60, 61, 62, 63, 64, 65, 66, 67, 68, 69, 70, 71, 72, 73, 74, 75, 76, 77, 78 and 79;
said nucleic acid molecule comprises an RNA strand comprising a nucleotide sequence selected from the group consisting of: SEQ ID NO: 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 and 250; or
said nucleic acid molecule comprises an RNA strand comprising a nucleotide sequence selected from the group consisting of: SEQ ID NO: 50, 51, 52, 53, 54, 55, 56, 57, 58, 80, 81, 82, 83, 84, 85, 86, 87, 88 and 89.

17-18. (canceled)

19. The nucleic acid molecule according to claim 1, wherein said cardiovascular gene target is Human diglyceride acyltransferase 2 (DGAT2).

20. The nucleic acid molecule according to claim 19 wherein:
said nucleic acid comprises an RNA strand comprising a nucleotide sequence selected from the group consisting of: 100, 101, 102, 103, 104, 105, 106, 107, 108, 109, 110, 111, 112, 113, 114, 115, 116, 117, 118 and 119;
said nucleic acid comprises an RNA strand comprising a nucleotide sequence selected from the group consisting of: 131, 132, 133, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169 and 170; or
said nucleic acid comprises an RNA strand comprising a nucleotide sequence selected from the group consisting of: SEQ ID NO: 90, 91, 92, 93, 94, 95, 96, 97, 98, 99, 120, 121, 122, 123, 124, 125, 126, 127, 128 and 129.

21-22. (canceled)

23. The nucleic acid molecule according to claim 1, wherein N-acetylgalactosamine is linked to either the antisense part of said inhibitory RNA or the sense part of said inhibitory RNA.

24. The nucleic acid molecule according to claim 23 wherein N-acetylgalactosamine is monovalent, divalent, or trivalent.

25. The nucleic acid molecule according to claim 23, wherein said nucleic acid molecule is covalently linked to an N-acetylgalactosamine molecule comprising the structure:

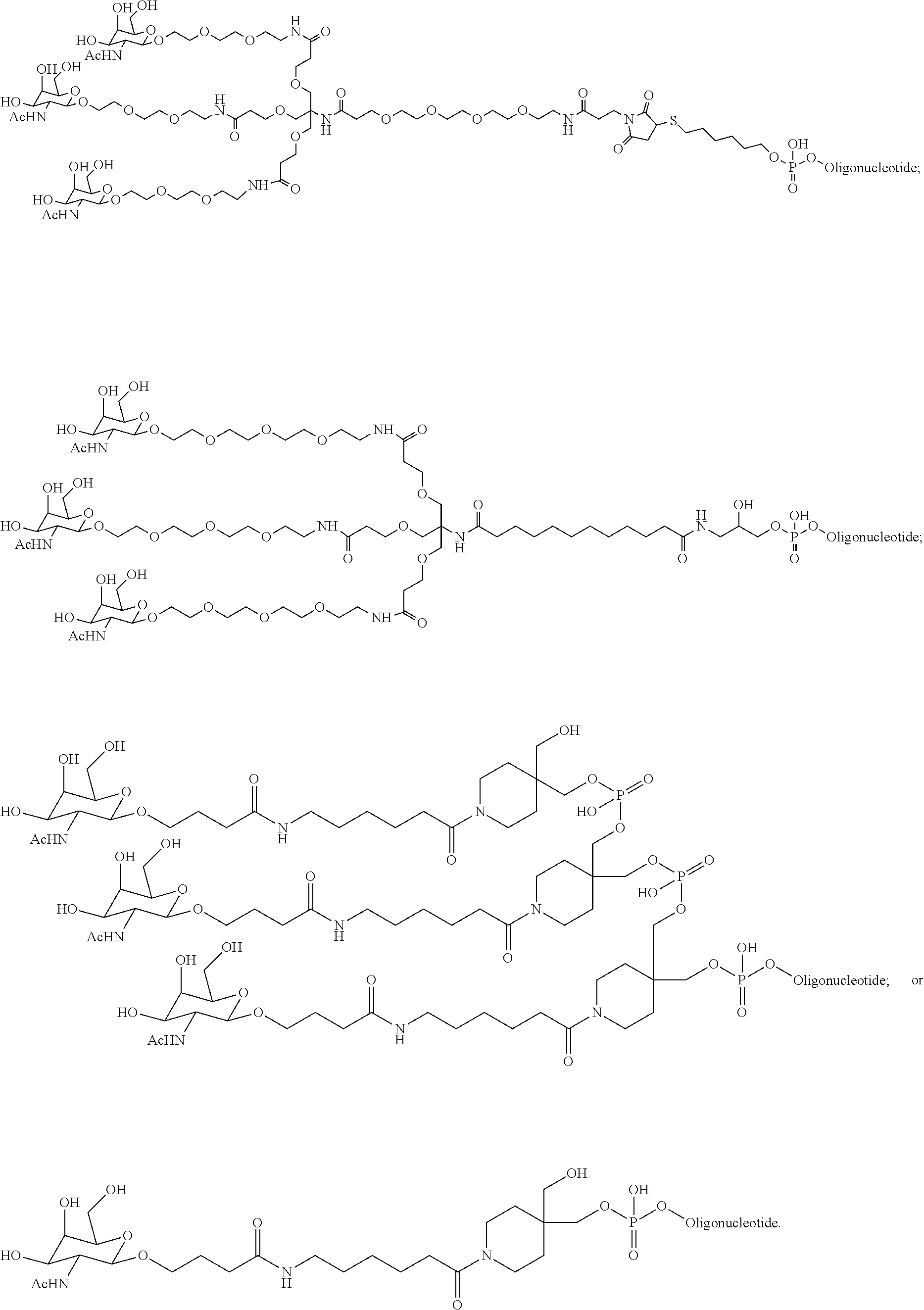

26-28. (canceled)

29. A pharmaceutical composition comprising at least one nucleic acid molecule according to claim 1.

30. (canceled)

31. The method according to claim 33, wherein hypercholesterolemia is familial hypercholesterolemia.

32. The method according to claim 33, wherein said disease associated with hypercholesterolemia is selected from the group consisting of: stroke, hyperlipidaemia, cardiovascular disease, atherosclerosis, coronary heart disease, aortic stenosis, cerebrovascular disease, peripheral arterial disease, hypertension, metabolic syndrome, type II diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

33. A method to treat a subject that has or is predisposed to hypercholesterolemia or a disease associated with hypercholesterolemia, comprising administering an effective dose of a nucleic acid according to claim 1, thereby treating the subject.
